(19) United States
(12) Patent Application Publication
(10) Pub. No.: US 2011/0159118 A1
(43) Pub. Date: Jun. 30, 2011

(54) Screening Method (Metabolite Grid) for Therapeutic Extracts and Molecules for Diabetes

(76) Inventor: Villoo Morawala Patell, Bangalore (IN)

(21) Appl. No.: 11/721,131

(22) PCT Filed: Dec. 8, 2000
(86) PCT No.: PCT/IB04/04077

§ 371 Date: Jun. 7, 2007

(51) Int. Cl. A61K 36/00; A61P 3/10

(52) U.S. Cl. 424/725

(57) ABSTRACT

The present invention provides therapeutic compositions derived from plants useful for treating diabetes. The present invention also provides methods for screening plant metabolites and constituents to identify therapeutic agents and/or dietary supplements for treating diabetes. The therapeutic compositions can be derived from plants such as Phyllanthus emblica, Azadiractha indica, Terminalia chebula, Mucunapuriens, Curcuma longa, Ficus glomerata, Phyllanthus niruri, Momordica charantia, Euphorbia royleana, Catharanthus roseus, Eugenia jambolana, Emblica officinalis, Gymnema sylvestre, Melia azadirechta, Morinda citrifolia, Pterocarpus marsupium, Tinospora cardifolia, Tribulus teristris, Trigonella foenum graecum, and Withania somnifera.

### FIELD OF THE INVENTION

The present invention relates to a plethora of selected plants, which were isolated and characterized for the therapeutically relevant molecules to be used in the treatment of Diabetes. The extracts from the plants were subjected to both, targeted and non-targeted screening procedures. The ongoing-targeted screening procedures, which feature a comprehensive metabolite profiling of multitudes of phyto-extracts, were envisaged to facilitate the creation of a metabolite grid. Extensive comparative analyses of the individual plant species with the existing drug and/or phyto-extract formulations in the market has revealed the presence of both, unique and common molecular constituents that will be used individually and/or in combination to accelerate the process of the discovery of novel therapeutic formulations. This invention also relates to an edible composition comprising of fifteen Indian herbal extracts which can be used as a dietary supplement and also useful in lowering the glucose levels in the blood of mammals, particularly humans, suffering from Diabetes mellitus.

### BACKGROUND OF THE INVENTION

Diabetes is often defined as a state in which homeostasis of carbohydrate and lipid metabolism is improperly regulated by insulin. This results primarily in elevated fasting and post-prandial blood glucose levels. If this imbalanced homeostasis does not return to normalcy and continues for a protracted period of time, it leads to a condition known as hyperglycemia that will in due course turn into a syndrome called Diabetes mellitus. The word Diabetes is derived from the Greek work “Diab”, meaning to pass through, namely, referring to the cycle of heavy thirst and frequent urination. Mellitus is derived from the Latin word “sweetened with honey” and alludes to the presence of sugar in the urine. Diabetes mellitus is the most common metabolic abnormality in the world. It is a metabolic disorder characterized by a lack of hormone insulin in the blood, which leads to abnormalities in the assimilation of carbohydrates in the body. Diabetes mellitus can be defined as a group of syndromes characterized by hyperglycemia, altered metabolism of lipids, carbohydrates and proteins along with increasing risks of complication from vascular diseases. Very often, the seriousness of the disease is realized on the development of secondary symptoms that manifest in many forms, namely, difficulty in healing of wounds, neuropathy and so on. It is estimated currently, that over 143 million people all over the world suffer from Diabetes mellitus and in the case of most people, suffering from Diabetes; it is not properly diagnosed until irreversible complications set in.

Diabetes has been known to be prevalent in India since the past few centuries and early recorded literature has even documented the physician, Sushruta's description of Diabetes around 1,000 B.C. and later on the physician, Charak. It is in fact believed that the knowledge of the syndrome of Diabetes mellitus, existed in India since pre-historic age. Its earliest reference (1,000 B.C.) in the Ayurvedic literature is found in mythological form, where it is said to have originated by eating Havisha, a special food that used to be offered at the time of Yagna organised by Daksha Prajapati (Kohli et al., Ayurvedic perspective of Diabetes and allied disorders, Vedic Life sciences).

However in developing countries like India, there is not much focus on Diabetes, given its chronic and slow nature. Moreover the economics of developing countries do not permit for devoting its resources to an understanding or search for remedies. The World Health Organisation has projected India, as the country with the fastest growing population of diabetics and it is estimated that between 1995-2025, Diabetes patients in India will increase by 195%.

Amongst the many classical, clinical symptoms associated with Diabetes, one which is typical, is an increase in the blood glucose, otherwise known as hyperglycemia, which, in turn may result in polyuria (frequent urination), polydipsia (excessive thirst), polyphigia (excessive hunger), weight loss and blurred vision, apart from glycosuria and acetone breath. The long terms complications arising out of untreated or ineffectively treated Diabetes include among others, retinopathy, nephropathy and peripheral neuropathy. Diabetes patients stand an increased risk of succumbing to cardiovascular diseases and strokes.

Recent developments in understanding the pathophysiology of the disease process have opened up several new avenues to identify and develop novel therapies to combat the diabetic plague. Phytochemicals identified from traditional medicinal plants present an exciting opportunity to develop new kinds of therapeutics. There is an urgent need to identify indigenous natural resources, procure them and study them in detail and their potential on newly identified targets in order to develop them as new therapeutics.

The increasing cost of modern treatment of Diabetes indicates a great need for the development of alternate strategies for the prevention and treatment of Diabetes. In rural pockets of developing countries, almost 90% of the population still rely on traditional medicines for their primary health care and an investigation into such traditional medicines have led to the discovery of at least 88 drugs. There is a need for a rationally designed interdisciplinary research programme, which could lead to the development of indigenous, renewable medicinal plant sources as practical and cost effective alternatives. It is believed that the therapeutic approach of several traditional medicinal systems is more holistic. The medicinal preparations from traditional medicines contain a variety of herbal and non-herbal ingredients that are believed to act on a variety of targets through various modes and mechanisms.

Since ancient times, traditional medicines all over the world have advocated the use of plants to treat Diabetes. The beneficial multiple activities like manipulating carbohydrate metabolism by various mechanisms, preventing and restoring integrity and function of beta cells, insulin releasing activity, improvement of the glucose uptake, utilisation and the anti oxidant properties present in the medicinal plants, which offers exciting opportunities, to develop them into novel therapeutics.

The multifactorial pathogenicity of Diabetes demands a multi-modal therapeutic approach. Thus, future therapeutic strategies require the combination of various types of multiple agents. Medicatrix naturae or the power of self-preservation or adjustment has been the motto of traditional medicinal practise, which prescribes poly herbal formulations. The theories of poly herbal formulation have the synergistic, potentiative, agnoistic/antognistic pharmacological agents within themselves due to the incorporation of plant medicines with diverse pharmacological actions. These pharmacological principles are known to work in a dynamic way to produce maximum therapeutic efficacy with minimum side effects. Traditional medicines ought not to be treated as a collection of therapeutic recipes. They are formulated and prepared, keeping in mind, the disease/sickness and the healing properties of the individual ingredients.

In the case of Diabetes, the main symptoms targeted were thirst, polyuria and glycosuria (Herbal Drugs as Antidiabetics: An Overview; Sanjay M. Jachak, CRIPS Vol. 3, Number 2, April-June 2002). There have been over 1200 plants that have been screened for activity on the basis of ethnopharmacology or on a random basis. Plant extracts have been tested under specific conditions as glucose-loaded, alloxan, streptozotocin or naturally diabetic subjects in various animals like rodents.

The Indian subcontinent is renown for its indigenous collection of natural remedies like Ayurveda, Unani, Siddha and so on, based on which, new therapeutic molecules can be obtained. A large number of drugs from plant sources are secondary metabolites, which have no role in plant metabolism, but are expected to play a significant role in plant defence mechanism. There is however, not much difference seen between the basic metabolic processes in plants and animals. There are approximately around 200 pure compounds from plant sources, which are reported to indicate a decrease in the blood glucose.

Over 1200 plant compounds worldwide have undergone tests at various levels in order to ascertain their ability to lower blood sugar levels and it was found that many of these compounds contained chemical components that possessed hypoglycemic activity, when, tested in either animal models or test tubes. However the research on such compounds involving human subjects has not brought much information to light so far. There have been a few herbal remedies for Diabetes tested on humans and these have revealed mild blood sugar lowering properties.

Type II Diabetes also known as non-insulin dependent Diabetes mellitus ('NIDDM') is more common than insulin dependent Diabetes mellitus, affecting 80-90% of all people affected with Diabetes. Initially NIDDM is often of gradual onset during the latter middle age. Later stages of this disease are very severe resulting in long term complications like kidney failure, heart problems, eye and nervous disease and other diseases as well. Obesity is an important factor in NIDDM. The symptoms can be vague and include fatigue, nausea, frequent urination and unusual thirst. NIDDM develops genetically in predisposed individuals. The pathological changes in the pancreatic islets of patients with NIDDM are not always apparent. Many patients are known to have normal to high plasma insulin levels. In such cases, very often, Diabetes does not arise from a shortage of insulin, but as a result of defects in the molecular machinery that mediates the action of insulin on its target cells. NIDDM is also caused by destruction of other mechanisms such as insulin resistance, down-regulation of insulin receptors, defects in insulin secretion from the pancreatic cells and other changes to the glucose transporter system. To date, no satisfactory method exists to treat or cure NIDDM without apparent toxicity to the patients. Therefore there is an urgent need to provide alternative treatments, effective in the prevention and/or treatment of NIDDM.

An ideal medication for the treatment and prevention of Diabetes would be one which would incorporate the following characteristics: ability to stimulate regeneration of pancreatic islets and beta cells responsible for insulin production and to increase c-peptide levels; ability to modulate the autoimmune destruction of the cells responsible for insulin production; ability to correct the dislipidemia associated with Diabetes; ability to decrease insulin resistance, with few or no side effects. However the known pharmaceutical compositions for treating Diabetes do not meet with this criterion. The pharmaceutical oral hypoglycemic agents produce inconsistent clinical results with frequent, severe side effects and there is a need felt for safe and effective oral hypoglycemic agents that provide the clinician with a wide range of options to prevent, treat and manage Diabetes since it has been held that the traditional drugs prescribed to balance blood sugar can result in serious liver damage and in some cases, even cause liver failure. Although many traditional plant treatments for Diabetes exist, it is only a few that have received scientific or medical scrutiny (Gray & Flatt Insulin-secreting activity of the traditional anti diabetic plant Viscum album (mistletoe)—Journal of Endocrinology, 1999).

Herbs have been frequently alluded to as part of ‘nature's pharmacy’. Even though herbal drugs act in more than one way analogous to modern drugs, herbal remedies are generally looked upon as a lot safer. In fact, many of the drugs used in conventional medicine are derived from herbs, instead of isolating the ‘active agent’. Herbalism uses the whole plant. In many cases it has been observed that plants contain constituents that work together synergistically and using the whole plant aids in decreasing the side effects that may occur when using isolated components.

Plants with medicinal properties, the gift of Mother Nature to mankind, have been in use in India over several centuries in the traditional systems of medicine like Ayurveda, Unani etc., for the treatment of diseases including Diabetes mellitus. They are considered to be effective and toxic. It is botanical medicine that provides a complete system of healing and prevention of the disease. It is the oldest and most natural form of medicine. Its history of efficacy and safety spans centuries and covers almost the entire planet. Since herbal medicine is holistic medicine, it is able to look beyond the symptoms to the underlying systemic imbalance and offers real and permanent solutions to the problems on hand.

Higher plants constitute one of our most important natural resources. They not only provide foodstuffs, fibers, and woods, but many biochemicals, such as oils, flavorings, dyes, and pharmaceuticals. Although plants are renewable resources, some species are becoming more difficult to obtain in sufficient amounts to meet increasing demands. Destruction of natural habitats and technical difficulties in cultivation too are responsible for the drastic reduction in plant availability. For example, it is claimed that a demand for paclitaxel, a potent anticancer compound, could endanger the forests of Taxus brevifolia (Pacific yew) because of the low paclitaxel content (40-100 mg/kg of bark) in and a slow growth of the trees.

For many natural chemicals it is possible to synthesize alternatives from petroleum, coal, or both. The economic limitations of chemical synthesis and the pollution that accompanies this type of chemical synthesis, however, have led to the development of cell culture and molecular engineering of plants for the production of important chemicals. Plant cell and organ cultures offer promising alternatives for the production of biochemicals since totipotency enables plant cells and organs to produce useful secondary metabolites in vitro. Molecular engineering of secondary metabolites has the potential to increase productivity and improve product composition.

The metabolism comprises a coordinate series of coupled enzymatic conversion in living organisms. The secondary metabolites are not vital to the cell death that produces them but contribute to the overall fitness of the organisms. The functions of these compounds in plants include protection against pests and pathogens. For man, plant secondary metabolites are useful as pharmaceutical dyes, fragrance, insecticides and/or flavours.

In order to regulate the biosynthesis of secondary metabolites, plants must accommodate their primary metabolic pathways. A coordinate regulation between these processes has been observed but the regulatory mechanisms are unknown. (Lelslie van der Fits & Johan Memelink, 2000). Production of secondary metabolites is controlled at the levels of expression of the biosynthetic genes by developmental tissue specific factors or by external signals. The accumulation of metabolites is induced by (methyl) jasmonate, a plant hormone produced in response to stress.

A biosynthesis of many classes of secondary metabolites in plants is induced by the stress hormone, jasmonate. The gene for ORCA-3, a jasmonate responsive APETALA2 (AP2) domain transcription factor was isolated by transferred DNA activation tagging and its over expression resulted in an enhanced expression of several metabolites biosynthetic genes and consequently, an increased accumulation of terpenoid Indole alkaloids. A regulation of metabolites biosynthetic genes by jasmonate responsive AP-2 domain transcription factors may link plant stress responses to changing metabolism. Plants can regulate primary metabolic pathways coordinately with secondary metabolism using a single transcription factor. Since the biosynthesis of many secondary metabolites is induced by jasmonate, the identification of an AP2 domain protein as a regulator of several genes involved in JA responsive metabolism uncovers a control mechanism that may be operative in other stress responsive plant metabolic pathways as well.

### PRIOR ART

U.S. Pat. No. 5,980,902 states that the leaves of Gymnema sylvestre, a herb belonging to the Asclepiadaecae family have been used by traditional medical practitioners of India to treat diabetic conditions for several centuries. Gymnema sylvestre has also been studied for its anti sweet properties, for its ability to inhibit small intestine absorption of glucose and for its ability to suppress increases in blood glucose levels following glucose administration. U.S. Pat. No. 5,900,240 refers to Syzgium cumini jamun, Gymnema sylvestre, Momordica charantia bitter gourd and Solanum melongena egg plant, the compositions of which will provide a herbal dietary supplement, which will be tolerated by insulin dependent diabetic sufferers without any undesirable side effects and which will allow blood glucose levels to be controlled to a level below that achievable by administration of insulin. European Patent number WO9842211 has claimed for nutritional supplements, which could be used as a treatment for poor glucose metabolism of Diabetes, and also for prevention of Diabetes by giving the metabolism a boost before the full-blown Diabetes develops. The purpose of Patent number JP4022627 has been to obtain an insulin secretagogue, useful as a curing agent for a patient suffering from hyperglycemia, ketoacidosis etc. instead of insulin.

CN1380072 patent states a medicine for preventing and curing diabetes, other metabolic disease and its complicating diseases. It includes extract of dried or fresh plant Gymnema sylvestre and voglepotang sugar, as compared with existent technology it has the advantages of high therapeutic effect, low toxic side effect and long acting time, not only can be used for preventing and curing various diabetes, but also can be used for preventing and curing hyperlipemia, adiposis, arteriosclerosis, X syndrome and other complicating diseases.

U.S. Pat. No. 5,980,902 describes compositions derived from Gymnema sylvestre leaf materials that may be administered orally, intravenously, subcutaneously or transdermally which are useful for treating patients having diabetes, impaired glucose tolerance, and various conditions associated with or symptoms of diabetes. Additionally, the compositions reduce polydipsia, polyuria and polyphagia, regenerate the pancreatic islets of Langerhans, including beta cells, increase endogenous insulin, lipase and amylase levels, increase production of proinsulin and c-peptide, and lower blood lipids and triglycerides and free fatty acids.

European patent number WO9510292 talks about glucose metabolism in a human patient being regulated by dosage forms that contain a naturally occurring, plant derived carbohydrate, an aqueous and water-miscible polar solvent extract, preferably an aqueous and ethanolic extract, of Gvmnema svlvestre in combination with a non-metabolizable polysaccharide preferably a Sterculia urens exudate, in a respective weight ratio in the range of about 1:2 to about 2:1.

Aqueous extracts from the leaves of G. svlvestre have been described as inhibiting temporarily the taste of sweet substances. It has also been reported that the raw leaves of G. sylvestre have been used in India as a folk medicine for various afflictions including diabetes mellitus. Some fourteen or fifteen different compounds are reported to have been isolated from the leaves of G. svlvestre by various techniques. Stocklin, J. Agr. Food Chem. 17(4):704-708 (1969); Sinsheimer, J. Pharm. Sci. 59(5):622-628 (1970). However, applicant is not aware of scientific information as to whether any of the noted chemicals, individually or collectively, contribute to the hypoglycemic properties. It has now been found, however, that the present aqueous and water-miscible polar solvent extract contains at least four fractions that are insulinotropic. Two of these fractions exhibit substantially equal and relatively strong insulinotropic activity.

Chinese patent CN1268515 relates to the extract of Gymnema svlvestre which is mainly composed of total triterpene saponin, flavone glycoside, anthocyan, polysaccaride, etc. in which the content of total triterpene saponin is 50-99%, and 25-40% of the total triterpene saponin are six kinds of new triterpene saponin compounds. Said extract possesses the active functions of lowering blood sugar, bloodfat and anti-thrombocyte coagulation.

U.S. Pat. No. 6,572,897 describes a composition that contains essential amounts of Alpha Lipoic Acid, Chromium, Lutein, Bioflavonoids (quercetin and rutin), Mormordica Charantia extract, Corosolic Acid, and Gymnema Sylvestre Extract, as well as other ingredients and healthy filler ingredients with clinical studies proven to assist in the maintenance of insulin sensitivity and healthy blood sugar levels.

U.S. Pat. No. 5,886,029 describes a medicinal composition including a pharmacologically significant quantity of (−)epicatechin augmented with a comparable amount of gymnemic acid useful for the treatment of diabetes in a human subject. The medicinal composition of the invention induces a significant reduction in serum glucose due to the regeneration of pancreatic islet cells. The unique combination of components in the medicinal composition leads to a regeneration of the pancreas cells, which then start producing insulin on their own. Since the composition restores normal pancreatic function, treatment can be discontinued after between about four and twelve months.

U.S. Pat. No. 5,137,921 describes the use of an inhibitory agent of an increase in blood sugar level, conduritol A obtained from dried leaves of Gymnema sylvestre or from dried bark of Marsdenia condurango by means of extraction.

Shamnugasundaram et al studied the use of Gymnema Sylvestre leaf extract in the control of blood glucose in Insulin-dependent Diabetes Mellitus, Journal of Ethnopharmacology 30, pp. 281-294, 1990.

Baskaran et al studied the antidiabetic effect of a leaf extract from Gymnema Sylvestre in Non-insulin-dependent Diabetes Mellitus, Journal of Ethnopharmacology 30, pp. 295-305, 1990. Patients were able to discontinue their conventional drug and maintain their blood glucose homeostasis with the extract of Gymnema Sylvestre(GS4) alone. These data suggested that the beta cells might be regenerated/repaired in Type 2 diabetic patients on GS4 supplementation. This is supported by the appearance of raised insulin levels in the serum of patients after GS4 supplementation.

Sugihara Y et al. described the antihyperglycemic effects of gymnemic acid IV, a compound derived from Gymnema sylvestre leaves in streptozotocin-diabetic mice, J Asian Nat Prod Res. 2000; 2 (4):321-7. Gymnemic acids derived from the methanol extract of leaves of Gymnema sylvestre, at doses of 3.4-13.4 mg/kg reduced the blood glucose levels by 13.5-60.0% 6 h after the administration, comparable to the potency of glibenclamide. These results indicate that insulin-releasing action of gymnemic acid IV may contribute to the antihyperglycemic effect by the leaves of G. sylvestre. Gymnemic acid IV may be an anti-obese and antihyperglycemic pro-drug.

Rathi et al. studied the effect of Gymnema sylvestre on protein-bound polysaccharide components & glycosaminoglycans in experimental diabetes. Indian J. Experimental Biol 19, pp. 715-721, 1981.

Shanmugasundaram et al. tried to find the possible effects of leaf extracts of Gymnema Sylvestre on the regeneration of the islets of langerhans in Streptozotocin-diabetic Rats. Journal of Ethnopharmacology 30, pp. 265-279, 1990.

CN1122699 describes capsules made from balsom pear, lagenaria peel, root of Chinese angelica, periwinkle etc. The clinical tests on more than 1000 cases of diabetes patients who do not rely upon insulin therapy verified that the total effective rate is up to 92.5%. It also has notable effect for reducing the blood sugar for diabetes patients relying upon insulin.

Singh S N et al. studied the effect of an antidiabetic extract of Catharanthus roseus on enzymic activities in streptozotocin induced diabetic rats, J Ethnopharmacol. 2001 August; 76(3):269-77. Extract at dose 500 mg/kg given orally for 7 and 15 days showed 48.6 and 57.6% hypoglycemic activity, respectively. Prior treatment at the same dose for 30 days provided complete protection against STZ challenge (75 mg/kg/i.p.x1). Results indicate increased metabolization of glucose in treated rats. Increased levels of lipid peroxidation measured as 2-thiobarbituric acid reactive substances (TBARS) indicative of oxidative stress in diabetic rats were also normalized by treatment with the extract.

United States Patent Application 20010021401 relates to an herbal therapeutic product for controlling diabetes mellitus comprising at least one hypoglycemic compound extracted from the pulp of a fruit from a species of genus Eugenia specifically Eugenia jambolina and a process for the preparation of the same.

US patent application 20020025349 describes a synergistic oral liquid herbal composition falling under the category of “Asavas” and “Arishtas”, useful for management of diabetes, said composition comprising a therapeutically effective amount of plant extracts selected from a. Momordica charantia (2-5%), b. Gymenma sylvestre (8-12%), c. Pterocarpus marsupium (8-12%), d. Eugenia jambolana (4-10%), and e. Trigonella foenum grecum (1-3%), and, optionally, comprising extracts/powder of Woodfordia fruticosa (2 to 5%), Piper longum (0.1 to 0.3%), Elettaria cardamomum (0.1 to 0.3%), Myristica fragrans (0.1 to 0.3%) and Ammomum subulatum (0.1 to 0.3%).

U.S. Pat. No. 5,972,342 describes mixtures isolated from grains of Eugenia Jambolana Lamarck, the preparation of such mixtures, the medicaments containing said mixtures or constituents of said mixtures, and the use of these mixtures for the treatment of diabetes and complications associated with Diabetes.

Kelkar in his article XP-000940531, Phytomedicine Volume 3 (4) pages 353-359, 1996/97, described a simple two step purification of antidiabetic compounds from Eugenia jambolana fruit-pulp; proteolytic resistance and other properties.

Rathi S S et al., assessed the efficacy of Momordica charantia (MC), Eugenia jambolana (EJ), Tinospora cordifolia (TC) and Mucuna pruriens (MP) in the prevention of murine alloxan dibetic cataract. (Phytother Res. 2002 December; 16(8):774-7). The incidence rate of cataract in MC, EJ, TC and MP treated groups at 120 days was only 0, 0, 1 and 2. Oral feeding of MC, EJ, TC and MP extracts for 1 month produced a fall of 64.33%, 55.62%, 38.01% and 40.17%, respectively, in the serum glucose levels in comparison with the 48 h level. After 2 months of treatment, the respective values were 66.96%, 59.85%, 40.41% and 45.63%. MC and EJ prevented the development of cataract while the protective effect was less with TC and MP along with a significant reduction of plasma glucose levels.

Grover J K et al., studied the amelioration of experimental diabetic neuropathy and gastropathy in rats following oral administration of plant (Eugenia jambolana, Mucuna pruriens and Tinospora cordifolia) extracts, Indian J Exp Biol. 2002 March; 40(3):273-6.

Anila L et al. studied the beneficial effects of flavonoids from Sesamum indicum, Emblica officinalis and Momordica charantia. Of the three sources, flavonoids isolated from Emblica officinalis exerted the maximum beneficial action by eliciting highly potent hypolipidaemic and hypoglycemic activities. (Phytother Res. 2000 December; 14(8):592-5)

Vikrant et al. tried to study the efficacy of the extracts of Momordica charantia and Eugenia jambolana to prevents hyperglycemia and hyperinsulinemia in fructose fed rats, J Ethnopharmacol. 2001 July; 76(2):139-43.

Sharma S B et al. studied the hypoglycaemic and hypolipidemic effect of ethanolic extract of seeds of Eugenia jambolana in alloxan-induced diabetic rabbits. J Ethnopharmacol. 2003 April; 85(2-3):201-6.

U.S. Pat. No. 6,551,627 15 describes an herbal medicinal composition for preventing or treating type II diabetes. The composition is comprised of extracts from Pterocarpus marsupium, Morus alba, Orthosiphon aristatus, Opiophogon japonicus, Rosa rugosa, Commelina communis, Trichosanthis kirilowii and Anemarrhena asphodeloides. The patent also describes the effect of the composition in reducing blood glucose level in a patient who has blood glucose level of about 200 to about 300 mg/dl at the beginning of treatment, increasing insulin secretion from pancreatic beta cells and a method of inhibiting degradation of complex carbohydrates to monosaccharides.

U.S. Pat. No. 6,448,450 talks about diphenylethylene Pterocarpus marsupium which when administered orally decreases blood glucose levels in rats. The compound is an effective anti-diabetic agent that can reduce abnormality of glucose metabolism in diabetes.

European patent application WO0172316 speaks about an edible Ayurvedic herbal composition for reducing blood sugar levels in humans, specially suffering from diabetes mellitus comprising a mixture of ingredients selected from the group consisting of Cinnamomum zeylanicum, Artocarpus heterophyllus, Salacia reticulata, Tinospora cordifolia and Pterocarpus marsupium. The mixture of the ingredients of the five selected herbs present in therapeutically effective proportions depending on the required strength of the mixture to treat abnormal levels of blood sugar and diabetes mellitus.

U.S. Pat. No. 6,562,791 describes a novel glucopyranoside, 6-hydroxy-2-p hydroxybenzylbenzofuran-7-C-.beta.-D-glucopyranoside isolated from Pterocarpus marsupium and to a process for the isolation thereof. The invention also relates to a pharmaceutical composition containing 6-hydroxy-2-p-hydroxybenzylbenzofuran-7-C-.beta.-D-glucopyranoside and to method for the treatment of diabetes using said compound.

Manickam M et al. studied the antihyperglycemic activity of phenolics from Pterocarpus marsupium, J Nat. Prod. 1997 June; 60(6):609-10. Of the 3 important, phenolic constituents of the heartwood of Pterocarpus marsupium, marsupsin (1), pterosupin (2), and pterostilbene (3), Marsupsin and pterostilbene significantly lowered the blood glucose level of hyperglycemic rats, and the effect was comparable to that of 1,1-dimethylbiguanide (metformin).

Sheehan E W discovered a constituent of Pterocarpus marsupium, (−)-epicatechin, as a potential antidiabetic agent. J Nat. Prod. 1983 March-April; 46(2):232-4. (−)-Epicatechin was found to reverse hyperglycemia in alloxan diabetic rats when given before or within 24 hr after the dose of alloxan. However, when doses of (−)-epicatechin (30 mg/kg, i.p., twice daily for 3 days) are begun 92 hr after alloxan, there is no significant difference in blood glucose levels between control and (−)-epicatechin treated rats.

Gupta S S et al. studied the effect of Tinospora cardifolia on fasting blood sugar level, glucose tolerance and adrenaline induced hyperglycaemia. Indian J Med. Res. 1967 July; 55(7):733-45.

Prince P S et al. studied the antioxidant activity of Tinospora cordifolia roots in experimental diabetes. J Ethnopharmacol. 1999 June; 65(3):277-81. T. cordifolia root extract (TCREt) (2.5 and 5.0 g/kg) for 6 weeks resulted in a decrease in the levels of plasma thiobarbituric acid reactive substances, ceruloplasmin and alpha-tocopherol in alloxan diabetic rats. The root extract also causes an increase in the levels of glutathione and vitamin C in alloxan diabetes. The root extract at a dose of 5.0 g/kg showed the highest effect. The effect of TCREt was more effective than glibenclamide.

Stanely P et al. studied the hypoglycaemic and other related actions of Tinospora cordifolia roots in alloxan-induced diabetic rats, J Ethnopharmacol. 2000 April; 70(1):9-15. Oral administration of an aqueous T. cordifolia root extract (TCREt) to alloxan diabetic rats caused a significant reduction in blood glucose and brain lipids. The extract caused an increase in body weight, total haemoglobin and hepatic hexokinase. The root extract also lowers hepatic glucose-6-phosphatase and serum acid phosphatase, alkaline phosphatase, and lactate dehydrogenase in diabetic rats.

Li M et al. studied the hypoglycemic effect of saponin from Tribulus terrestris, Zhong Yao Cai. 2002 June; 25(6): 420-2. The level of serum glucose could be significantly reduced by saponin from Tribulus terrestris, which was the rate of 26.25% and 40.67% in normal mice and diabetic mice in respectively. The level of serum triglyceride could be reduced 23.35%.

U.S. Pat. No. 6,042,834 describes a herbal composition for the treatment of diabetes, comprising 15 percent by weight of dried, powdered seeds of Trigonella foenum-graecum; 23 percent by weight of dried, powdered seeds of Nigella sativa; 10 percent by weight of dried, powdered leaves of Origanum vulgare; 10 percent by weight of dried, powdered sap of Rosmarinus officinalis; 15 percent by weight of dried, powdered beans of Lupinus tennis; 12 percent by weight of dried, powdered black leaves of Lawsonia inermis; and 15 percent by weight of dried, powdered seeds of Foeniculum vulgare.

An Indian patent application 305/MAS/99 describes a process for preparation of an antidiabetic herbal drug from the plants trichopus zeylanicus, withania somnifera and piper longum.

Andallu B et al studied the hypoglycemic, diuretic and hypocholesterolemic effect of winter cherry (Withania somnifera, Dunal) root, Indian J Exp Biol. 2000 June; 38(6):607-9. Hypoglycemic, diuretic and hypocholesterolemic effects of roots of W. somnifera (ashvagandha) were assessed on human subjects. Decrease in blood glucose was comparable to that of an oral hypoglycemic drug. Significant increase in urine sodium, urine volume, significant decrease in serum cholesterol, triglycerides, LDL (low density lipoproteins) and VLDL (very low density lipoproteins) cholesterol were observed indicating that root of W. somnifera is a potential source of hypoglycemic, diuretic and hypocholesterolemic agents.

U.S. Pat. No. 5,470,879 describes a process for stimulating the secretion of insulin and for the treatment of non-insulin dependent diabetes by the administration of effective quantities of substantially pure 4-hydroxyisoleucine or its lactone form or mixtures thereof obtained from Trigonella foenum graecum L.

Vats V et al. evaluated the anti-hyperglycemic and hypoglycemic effect of Trigonella foenum-graecum Linn, Ocimum sanctum Linn and Pterocarpus marsupium Linn in normal and alloxanized diabetic rats, J Ethnopharmacol. 2002 January; 79(1):95-100.

Abdel-Barry J A et al. studied the hypoglycemic effect of aqueous extract of the leaves of Trigonella foenum-graecum in healthy volunteers, East Mediterr Health J. 2000 January; 6(1):83-8.

Ali L et al. characterized the hypoglycemic effects of Trigonella foenum graecum seed, Planta Med. 1995 August; 61(4):358-60. The whole powder of Trigonella foenum graecum seeds and its extracts were tested for their hypoglycemic effect on normal and diabetic model rats. The powder, its methanol extract, and the residue remaining after methanol extraction had significant hypoglycemic effects when fed simultaneously with glucose.

Zia T et al. evaluated the oral hypoglycaemic effect of Trigonella foenum-graecum L. (methi) in normal mice, J Ethnopharmacol. 2001 May; 75(2-3):191-5. The presence of hypoglycemic activity in aqueous and methanolic extract indicates that the active compounds are polar in nature.

Gupta A et al. studied the effect of Trigonella foenum graecum (fenugreek) seeds in glycemic control and insulin resistance in type 2 diabetes mellitus: a double blind placebo controlled study. J Assoc Physicians India. 2001 November; 49:1057-61. Adjunct use of fenugreek seeds improves glycemic control and decreases insulin resistance in mild type-2 diabetic patients. There is also a favorable effect on hypertriglyceridemia.

Sharma R D et al. studied the effect of fenugreek seeds on blood glucose and serum lipids in type I diabetes, Eur J Clin Nut. 1990 April; 44(4):301-6. The fenugreek diet significantly reduced fasting blood sugar and improved the glucose tolerance test. There was a 54 percent reduction in 24-h urinary glucose excretion. Serum total cholesterol, LDL and VLDL cholesterol and triglycerides were also significantly reduced. The HDL cholesterol fraction, however, remained unchanged. These results indicate the usefulness of fenugreek seeds in the management of diabetes.

In U.S. Pat. No. 6,391,854, a water-soluble fraction of Momordica charantia and methods for its preparation and use in the treatment of hyperglycaemic disorders, is provided for. In Patent number R0116044 patent application, the process of extracting the immature, dried, milled fruit of Momordica charantia with methanol and concentrating it in an ethanolic solution till a viscous product is obtained, is a process for preparing a medicine for internal use and used as an adjuvant in the therapy of Diabetes mellitus. In U.S. Pat. No. 6,127,338, a water-soluble extract of Momordica charantia, methods for its preparation and use in the treatment of hyperglycaemic disorders are provided and so also in Patent number WO9843484.

In U.S. Pat. No. 6,043,824, a herbal composition for the treatment of Diabetes comprising therein of Trigonella foenum-graecum, Nigella sativa, Origanum vulgare, Rosmarinus officinalis, Lupinus termis, Lawsonia inermis and Foeniculum vulgare in a capsule form has been claimed. U.S. Pat. No. 5,886,029 alludes to a medicinal invention, which results in a significant reduction in serum glucose due to the regeneration of pancreatic islet cells. The medicinal composition includes Gymnemic acid, Cinnamomum tamala, Syzgium cumini, Trigonella foenum graceum, Azardichta indica, Ficus racemosa and Tinospora cordifolia. This combination leads to a regeneration of pancreas cells, which then start producing insulin on their own.

Patent number WO0172316 describes an edible ayurvedic herbal composition for reducing the blood sugar level in humans suffering from Diabetes mellitus comprising therein, a mixture of ingredients selected from Cinnamomum zeylanicum, Artocarpus heterophyllus, Salacia reticulata, Tinospora cordifolia and Pterocarpus marsupium in therapeutically effect proportions to treat abnormal levels of blood sugar and Diabetes mellitus.

U.S. Pat. No. 5,917,052 states that no prior study has described any hypoglycaemic activity or extracts of Cryptolepis sp or that quindoline alkaloids such as cryptolepine or quindoline would be useful as hypoglycaemic agents. The invention provides a method for the use of extracts from Cryptolepis sp and compounds of the quindoline family of alkaloids such as quindoline, cryptolepine etc. as well as pharmaceutically acceptable salts thereof as hypoglycaemic agents or as agents to lower triglyceride levels, particularly in diabetic subjects.

In U.S. Pat. No. 5,837,255, it has been held that no prior study has described any hypoglyemic activity of extracts of Harungana spp or Vismia spp nor was there any prior suggestion that anthracenone compounds such as harunganin or vismin are useful as hypoglycaemic agents. The invention provides a method for the use of extracts from Harunganin spp or from Vismia spp and for the use of anthracenone compounds harunganin and vismin, as well as pharmaceutically acceptable salts thereof as hypoglycaemic agents or agents to lower blood glucose levels, particularly in diabetic subjects.

U.S. Patent number 2002041904 has mentioned that in recent years among therapeutic drugs classified as anti diabetic agents, alpha glucosidase inhibitors which inhibit the activity of alpha-glucosidase have been widely used in the treatment of Diabetes and pre-Diabetes. Salacia reticulata has been used since ancient times in the ancient medicines of India and Sri Lanka. The object of this invention has been to provide a novel compound which is extracted from the woody climbing plants, Salacia prinoides and Salacia oblonga and is superior in terms of its characteristic of inhibiting the activity of alpha-glucosidase (compound being referred to as ‘alpha-glucosidase inhibitor’). In U.S. Pat. No. 5,691,386, it has been reported that plant of the genus Salacia has been used to treat Diabetes. In India, a hot water extraction of the whole plant Salacia prinoides has been taken orally as an anti-diabetic (P. N. Mehra et al., Res. Bull Punjab University Sci 20: 487-502 (1969). In Sri Lanka aqueous extracts of the roots of Salacia reticulata have been used in the treatment of Diabetes mellitus (E. H. Karunanayake et al., J. Ethnopharmacol 13 (2): 227-228 (1985). This invention claims to provide a novel triterpenoid compound, 3-beta, 30-dihydroxylup-20-29-en-2 one as well as pharmaceutically acceptable salts thereof, having hypoglycaemic activity, hypoglycemic compositions comprising the novel triterpenoid compound in purified form as well as methods for their use, as an hypoglycaemic agent. The invention further encompasses compositions comprising the triterpenoid compounds in purified form or pharmaceutically acceptable salts for use as hypoglycaemic agent, useful for the treatment of Diabetes.

U.S. Pat. No. 5,916,567 relates to a herbal therapeutic product for treating Diabetes and relates to a therapeutic product processed from the seed of a plant from the family Leguminosea, whose fibres affect the blood sugar level by increasing the viscosity of the unstirred layer between food and the lining of the intestines and the stomach thereby making the carbohydrates available for absorption at a slower rate.

U.S. Pat. No. 5,470,873 discloses a composition for the treatment of NIDDM comprising therein, maltol, obtained from Ginseng roots and an extract obtained from Orthosiphon aristatus, effective in regulating blood glucose levels in diabetic animals but this by itself is not sufficient to normalise the glucose levels, nor was any lasting effect determined following termination of the treatment.

The present invention relates to an amalgam of 15 (Fifteen) different traditional Indian medicinal plants, the compounds of which have been identified and isolated in order to have the same screened to ascertain their therapeutic effects in treating Diabetes, more specifically type II Diabetes with the compositions derived from non-toxic medicinal plants and effective in the prevention, treatment and cure of NIDDM.

### BRIEF DESCRIPTION OF THE TABLES AND FIGURES

Table 1 gives the list of medicinal plants used to arrive at the formulation, AGT_D_For_{—}0001

Table 2 gives the list of extracts from individual plants.

Table 3 shows a representative mass spectral peak grid arrived at using a comparative mass spectral peak analysis approach that illustrates the components that are either common or unique to individual extracts.

Table 4 shows the comparative results of the mass spectrometry analysis of the chloroform-methanol fractions of formulation AGT_D_For_{—}0001_{—}05:03 and its constituents, AGT_D_Mch_Se_{—}05:03, AGT_D_Tfg_Se_{—}05:03, AGT_D_Ej_Se_{—}05:03 & AGT_D_Gs_Ml_{—}05:03 indicating the common mass spectral peaks occurring both in the formulation and the constituent plants masses (m/z)

Table 5 shows the comparative results of the mass spectrometry analysis of the methanolic fractions of formulation AGT_D_For_{—}0001_{—}05 and its constituent plants, AGT_D_Mch_Se_{—}05, AGT_D_Tfg_Se_{—}05, AGT_D_Ej_Se_{—}05 and AGT_D_Gs_Ml_{—}05.

Table 6 fives an estimate of total number of constituents contained in a few of the individual plant extracts that have been characterized using HPLC-based metabolite fingerprinting.

Table 7 summarizes the comparative HPLC-based fingerprinting analysis done in the case of different plant extracts using a few representative instances (Ma_Le_Wa_{—}01 v/s Ws_Ro_Wa_{—}01; Ca_Ro_Et_{—}01 v/s Ws_Ro_Et_{—}01 and Ej_Se_Me_{—}01 v/s Tt_Fr_Me_{—}01).

FIG. 1 (A-F) gives the representative mass spectra of methanolic extracts (AGT_D_Gs_Ml_{—}05, AGT_D_Tfg_Se_{—}05, AGT_D_Ej_Se_{—}05) and chloroform extracts (AGT_D_Gs_ML_{—}03, AGT_D_Tfg_Se_{—}03, and AGT_D_Ej_Se_{—}03)

FIG. 2 (A-E) gives the representative mass spectra of the comparative mass spectrometric analysis between the chloroform fractions of the formulation, AGT_D_For_{—}0001_{—}03 and the chloroform extracts of a few of its constituents, AGT_D_Mch_Se_{—}03, AGT_D_Ej_Se_{—}03, AGT_D_Tfg_Se_{—}03 and AGT_D_Gs_Ml_{—}03.

FIG. 3 (A-E) gives the representative mass spectra of the comparative mass spectrometric analysis between the chloroform-methanol fractions of the formulation, AGT_D_For_{—}0001_{—}05:03 and the chloroform extracts of a few of its constituents, AGT_D_Mch_Se_{—}05:03, AGT_D_Ej_Se_{—}05:03, AGT_D_Tfg_Se_{—}05:03 and AGT_D_Gs_Ml_{—}05:03.

FIG. 4 (A-E) gives the representative mass spectra of the comparative mass spectrometric analysis between the methanol fractions of the formulation,

AGT_D_For_{—}0001_{—}05 and the methanol extracts of a few of its constituents,

### AGT_D_Mch_Se_{—}05, AGT_D_Ej_Se_{—}05, AGT_D_Tfg_Se_{—}05 and AGT_D_Gs_Ml_{—}05.

FIG. 5 gives the chromatograms of extracts Ma_Le_Wa_{—}01 and Ws_Ro_Wa_{—}01 at selected wavelengths

FIG. 6 gives the chromatograms of extracts Cr_Ro_Et_{—}01 and Ws_Ro_Et_{—}01 at selected wavelengths

FIG. 7 gives the chromatograms of extracts Ej_Se_Me_{—}01 and Tt_Fr_Me_{—}01 at selected wavelengths

### DESCRIPTION

The object of the invention is to provide edible herbal dietary supplements. The invention relates to a method and a composition for potentiating insulin activity to treat Diabetes patients. This composition has an effect on smoothing out fluctuations in the glucose levels.

The insulin potentiating agents are natural substances derived from plant extracts and can be safely consumed by humans. This naturally derived agent has an advantage in that it does not cause side effects. These agents can be used with conventional drug treatments like oral hypoglycemic agent or insulin.

One of the main problems that have been reported in the case of phyto-formulations is the lack of clarity in terms of comprehensive qualitative and quantitative characterization of all the detectable components present in the mixture. The availability of such information about phyto-extracts will play a major role in the scientific validation and standardization of both the therapeutic effects and constituents present in these phytoextracts.

Metabolite profiling has emerged as a robust tool that is fast, reliable, sensitive and suitable for automation, covering a significant number of metabolites. A range of analytical technologies enhances the sensitivity and universality of mass spectrometry by chromatographic separations. Although the use of multi-target profiling had been earlier limited to rapid clinical detection of human diseases, metabolic screening approaches using mass spectrometry are being increasingly used in plant research at present. A major advantage of mass-spectrometry is that unknown peaks can be determined as reliably as known target analytes without prior knowledge of their exact chemical structure. Studies using gas chromatography/mass spectrometry (GC/MS) have automatically quantified 326 distinct compounds from Arabidopsis thaliana leaf extracts. It has been possible to assign a chemical structure to approximately half of these compounds. Comparison of four Arabidopsis genotypes (two homozygous ecotypes and a mutant of each ecotype) showed that each genotype possesses a distinct metabolic profile. Data mining tools such as principal component analysis enabled the assignment of “metabolic phenotypes” using these large data sets. The results of this study have shown that metabolic phenotypes of the two ecotypes were more divergent than were the metabolic phenotypes of the single-loci mutant and their parental ecotypes. These results demonstrate the use of metabolite profiling as a tool to significantly extend and enhance the power of existing functional genomics approaches. Due to the increased chemical complexity and diversity at the metabolite level in higher plants, no singular technique exists for profiling all cellular metabolites concurrently. This problem can be approached through the division of metabolites into major profiling classes, i.e. triterpenoids, phenolics, lipids, carbohydrates, amino acids and carbohydrates. The hyphenated mass spectrometric techniques such as GC/LC/ESI− MS provide both relative quantitative abundances and specific information that can be utilized in chemical identification. Methods have been developed using HPLC interfaced with an ion trap mass spectrometer capable of sequential tandem mass spectrometry for profiling plant metabolites, i.e. HPLC-ESI-MSⁿ. This approach has been used to profile saponin glycosides in multiple cultivars of alfalfa followed by the comparison of these profiles to the model legume M. truncatula. To date, twenty-seven novel saponin glycosides in M. truncatula have been identified using this technology. This technology was also used to identify novel malonated saponin glycosides in alfalfa and M. truncatula.

### Metabolite Grid (Metagrid)

The plants selected for the isolation of therapeutically relevant extracts/molecules (“Yezdex”) to be used in the treatment of Diabetes, are being subjected to both targeted and non-targeted screening procedures. The ongoing-targeted screening procedures, which feature a comprehensive metabolite profiling of multitudes of phyto-extracts, were envisaged to facilitate the creation of a metabolite grid. Extensive comparative analyses of the individual plant species with the existing drug and/or phytoextract formulations in the market has revealed the presence of both unique and common molecular constituents that can be used individually and/or in combination to accelerate the process of discovery of novel therapeutic formulation. (FIG. 8)

### Screening Methodologies:

### Extraction: (See FIGS. 9 & 10)

Method 1: The successive extraction from various medicinal plants parts was carried out using soxhlet extractor. The solvents used, were based on their sequential polarity starting from non-polar to polar, wherein, various classes of metabolites were extracted viz; petroleum ether (phytosterols, fixed oils and fats), benzene (fixed oils and fats), chloroform (alkaloids), acetone (phytosterols, phenolics and tannins) ethanol (alkaloids, carbohydrates, glycosides, phytosterols, saponins, phenolics, tannins, proteins and amino acids) and water (alkaloids, carbohydrates, glycosides, saponinns, phenolics, tannins, proteins, amino acids, gums and mucilage) at 65° C. These fractions were lyophilized and stored in amber colored bottles at 4° C.

Phytochemical investigations were also carried out on these extracts using various tests like Mayer's and Dagendorf's tests for alkaloids; Molisch, Fehling and Benedict tests for carbohydrates; Lieberman Buchard's test for phytosterols and triterpenes; spot test for fixed oils and fats; Ferric chloride and Lead acetate test for phenolic compounds and tannins; Ninhydrin and Biuret tests for protein and aminoacids; alcoholic precipitation followed by Molisch test for gum and mucilages.

Method 2: To characterize a particular class of metabolites, fractional extraction procedure was adopted. In this method, various metabolite classes were screened like polysaccharides, terpenoids, phenolics, alkaloids, oils, fat and waxes present in the medicinal plants parts. (FIG. 11)

### High Performance Liquid Chromatography (“HPLC”) Profiling:

The extracted fractions were subjected to HPLC using μ bondapak C₁₈ column (Waters Alliance 2695 Separation Module) to separate the constituent metabolites. The fractions were eluted using a combination (80:20, 60:40, 50:50, 40:60, 20:80) of methanol:water/acetonitrile:water. The gradient run was also carried out wherever required. 5-10 ul of sample was injected with flow rate of 1 ml/min and HPLC run was performed for 30 minutes. The detection was carried out on photodiode array and the analysis of the results was done with the help of Millennium™ software.

### Identification and Characterization of Purified/Partially Purified Extracts by MALDI TOF:

The metabolites were identified and characterized by using the MALDI-TOF Voyager system 4266. The matrix for MALDI-TOF used was alpha cyano-4-hydroxycinnamic acid. Nuclear Magnetic Resonance (NMR) will be performed for unique and common fallouts of Metagrid for it structure elucidation.

### Comprehensive Constituent Profiling and Creation of the Metagrid:

A comparative profile of a therapeutic formulation and its individual constituents has been worked upon. AGT_D_For_{—}0001 (See Table 1), a therapeutic formulation, which comprises of approximately 15 (Fifteen) medicinal plants and their comparative analyses has been undertaken using mass spectrometry (MALDI-TOF MS). In addition to the formulation, 51 (Fifty One) individual plant extracts (see Table 2) have been comprehensively profiled using HPLC. Representative analytical data of the biochemical profiling carried out thus far is shown below:

### Mass-Spectrometry Based Comprehensive Constituent Profiling of the Formulation AGT_D_For_{—}0001 and the Constituent Plants

### Example 1

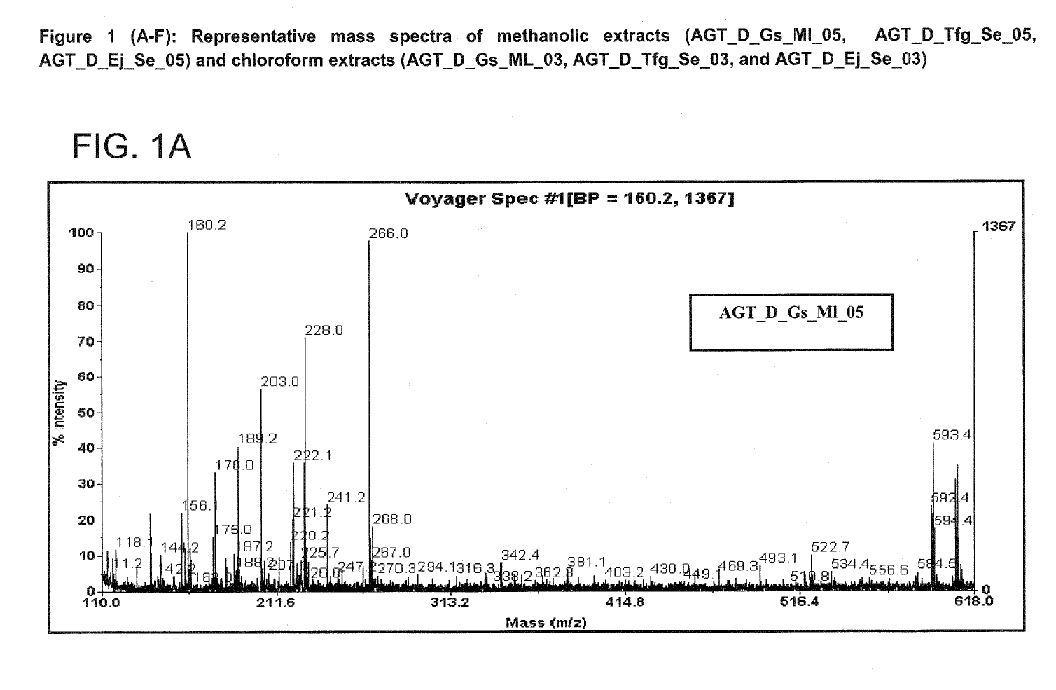

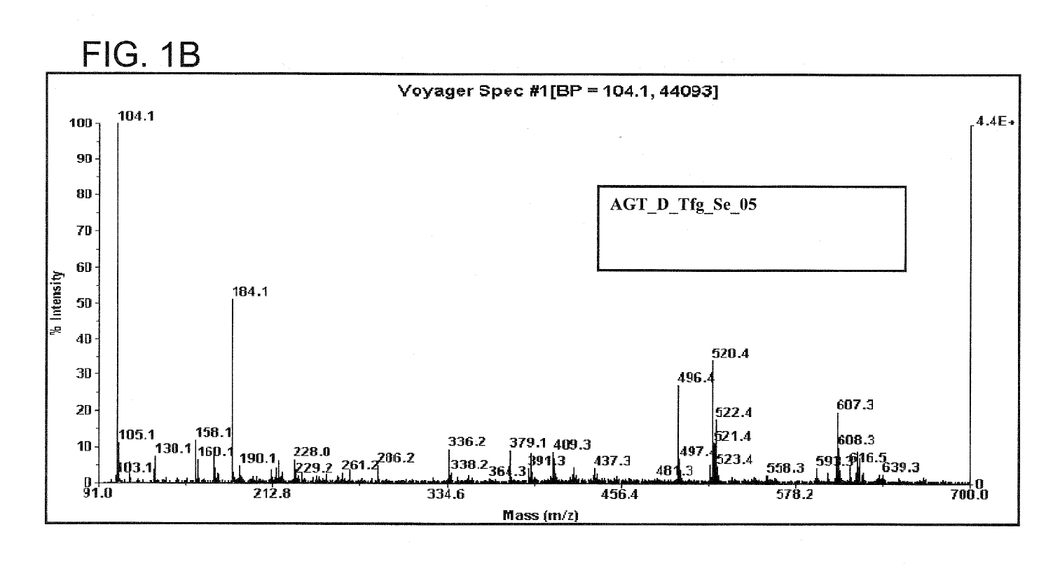

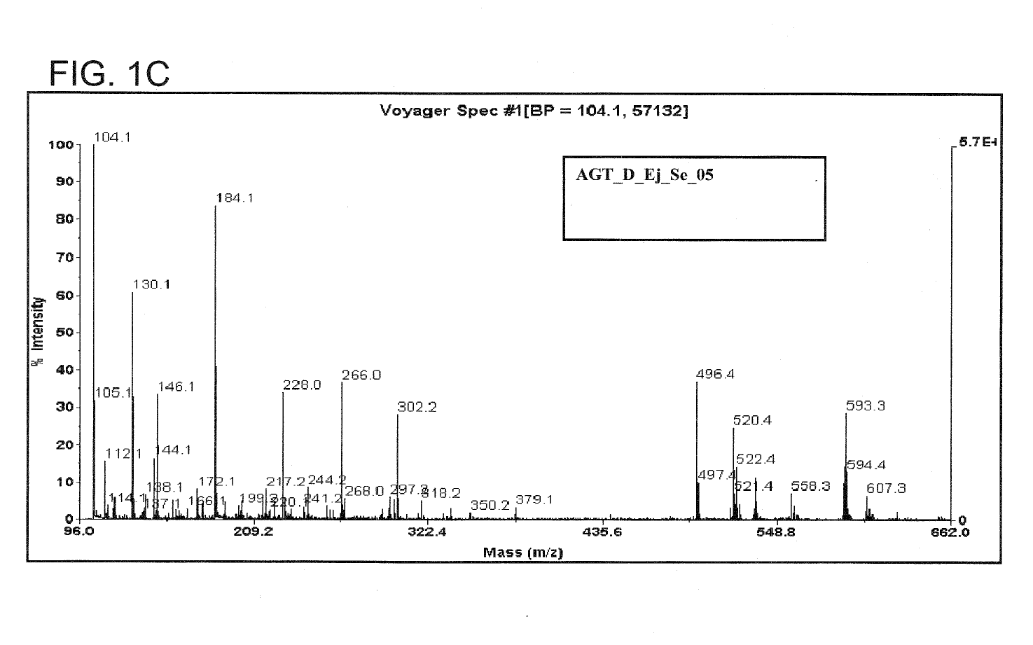

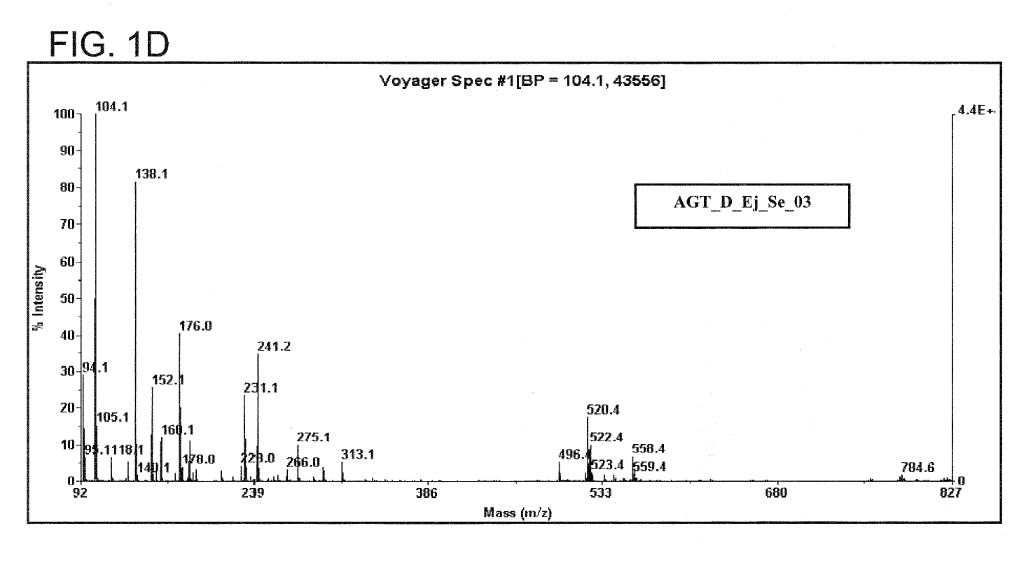

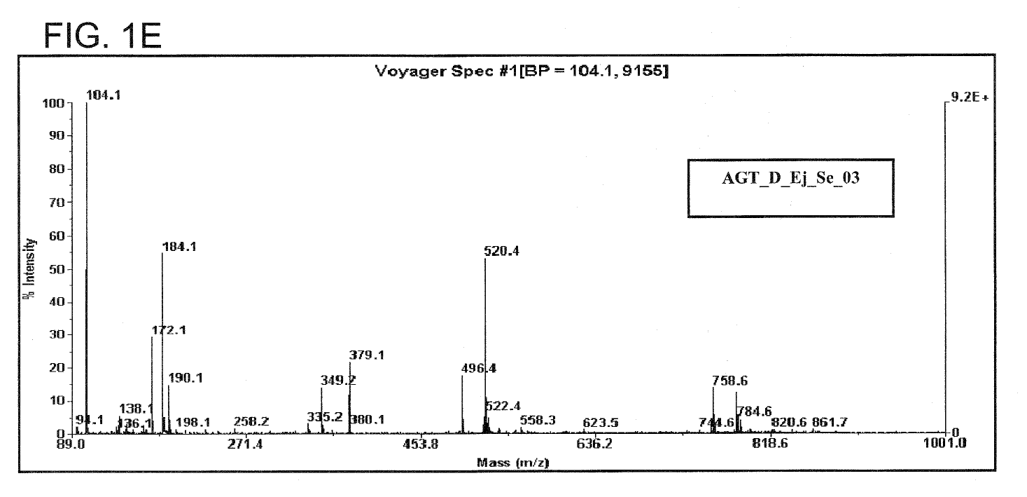

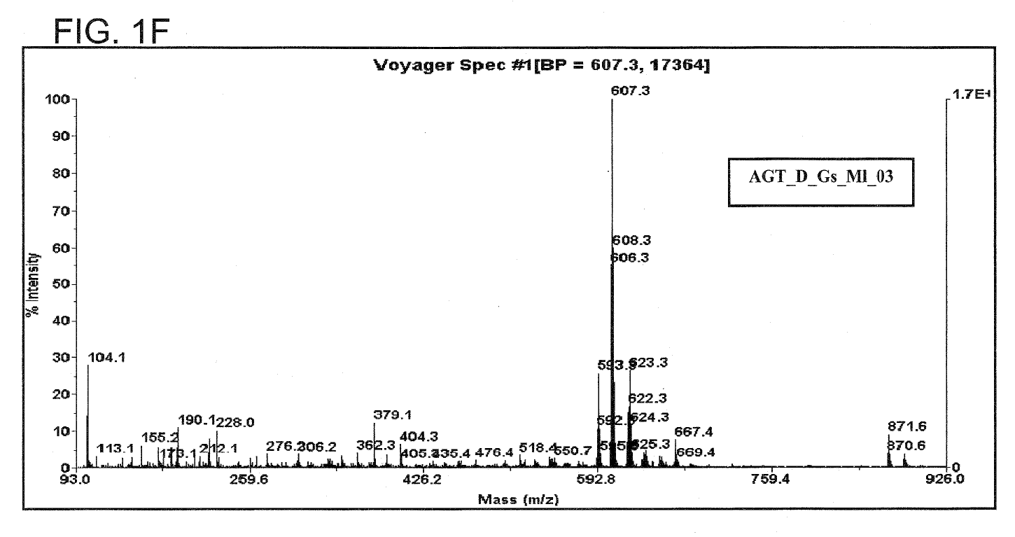

The methanolic and ethanolic extracts of Tfg, Ej and Gs were analysed using the screening method 1 as described earlier. The methanolic extracts AGT_D_Gs_Ml_{—}05, AGT_D_Tfg_Se_{—}05, AGT_D_Ej_Se_{—}05, and chloroform extracts AGT_D_Gs_ML_{—}03, AGT_D_Tfg_Se_{—}03, AGT_D_Ej_Se_{—}03 when compared, revealed common mass spectral peaks in both the solvents, viz. m/z 104, 112, 176, 184, 212, 228, 241, 496, 522, 592, 606. (As shown in Table 3 and FIG. 1) It was also observed that molecular mass spectral peaks m/z 203, 267, 337, 634, 694 were unique to the methanolic extracts and molecular mass spectral peaks m/z 190, 336, 340, 390, 520, 621 were unique to the chloroform extracts.

### Example 2

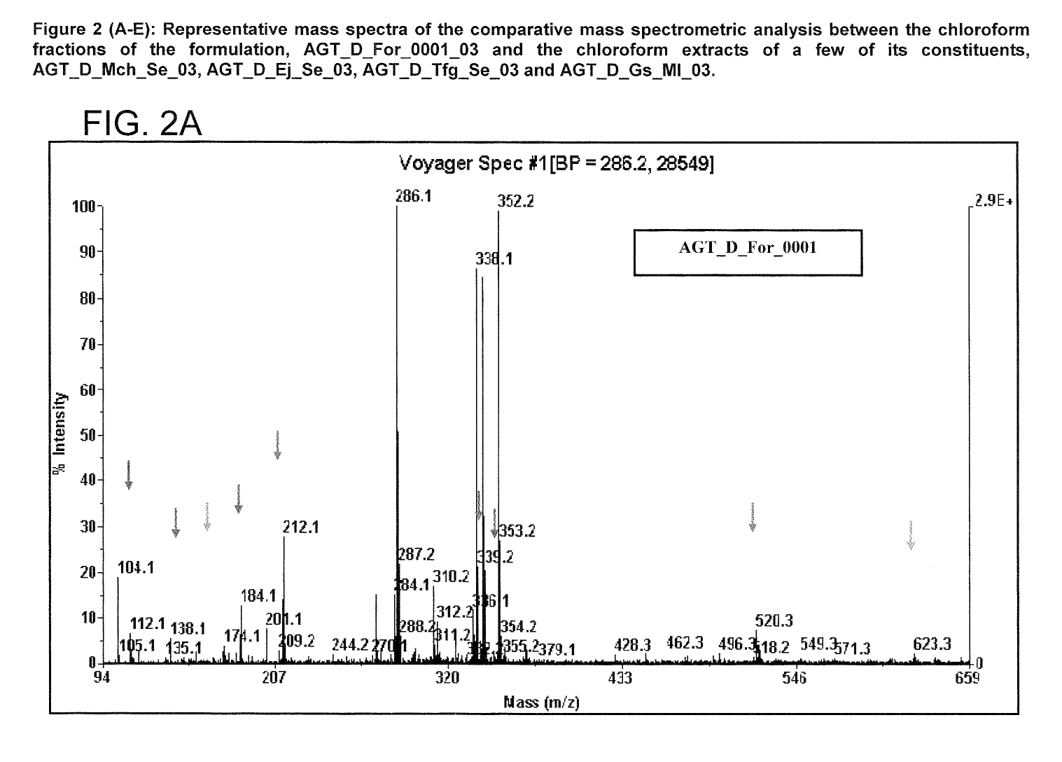

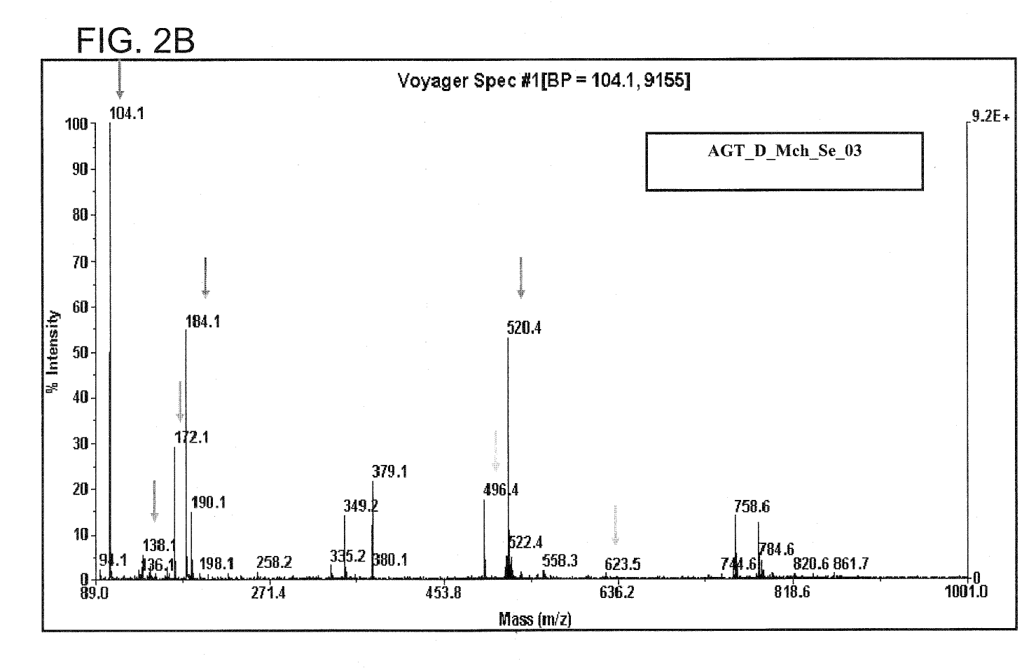

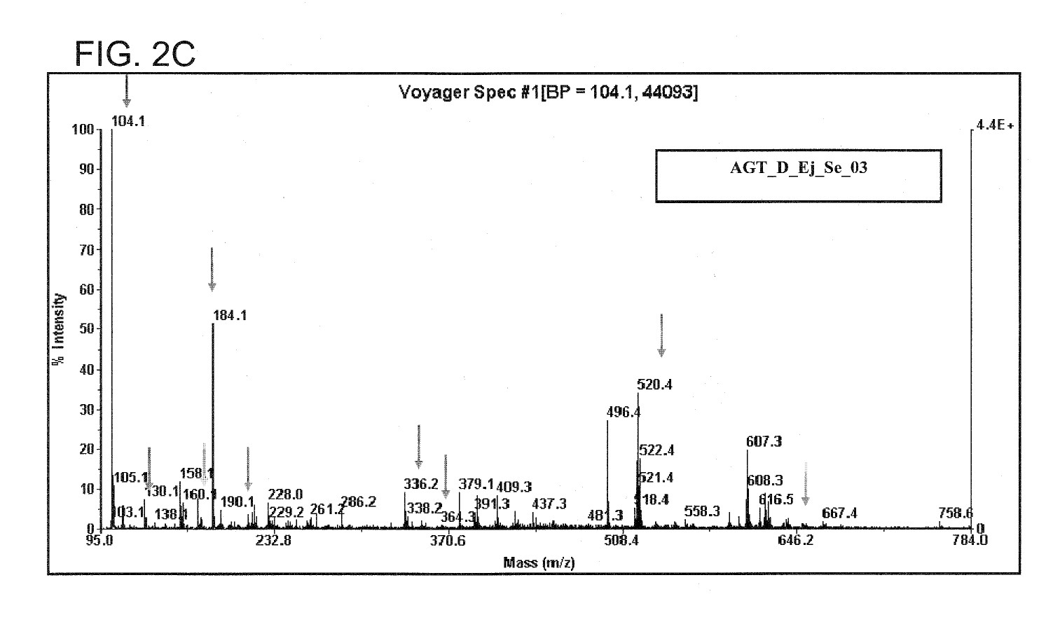

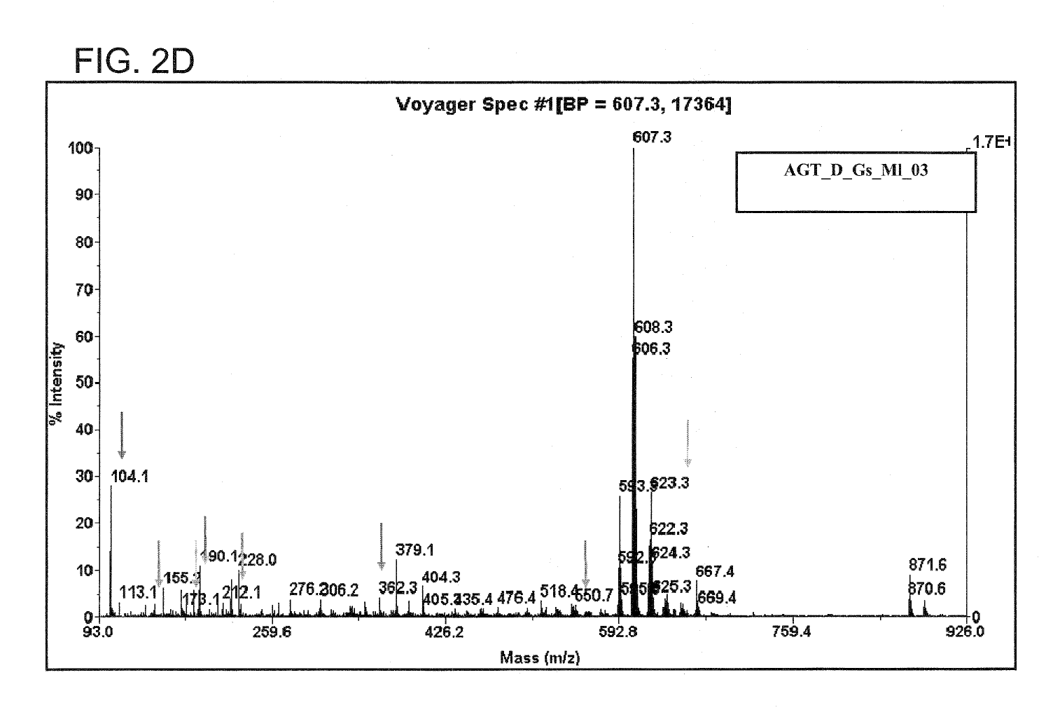

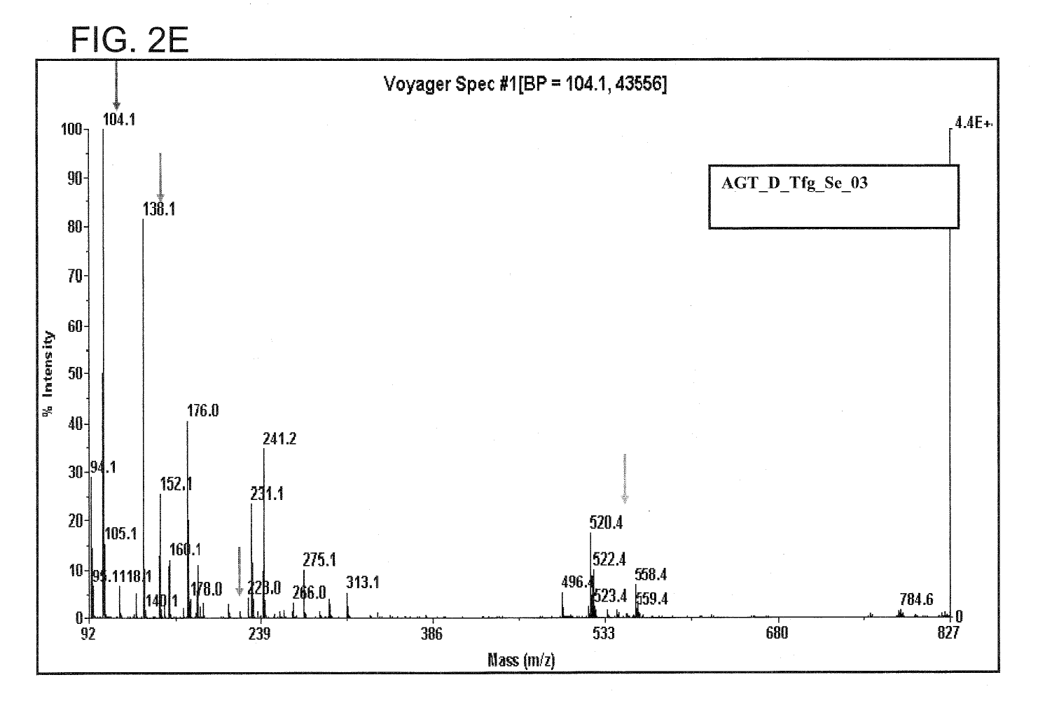

A comparative profiling of the therapeutic formulation, AGT_D_For_{—}0001, and its individual constituents, mentioned supra, was carried out to isolate terpenoids/phenolics, using method 2 as previously described. The comparative results of the mass spectrometry analysis between the chloroform fractions of the formulation, AGT_D_For_{—}0001_{—}03 and its constituents, AGT_Mch_Se_{—}03, AGT_D_Ej_Se_{—}03, AGT_D_Tfg_Se_{—}03, AGT_D_Gs_Ml_{—}03 revealed a few common mass spectral peaks m/z 104, 138, 172, 184, 336, indicating the presence of terpenoids/phenolics which may play a significant role in the treatment of diseases. The mass spectral peaks such as m/z-212, 288, 338, 496.3, 520.39, 623.48 that are present in formulation but are also uniquely present in few of the medicinal plant parts analysed thus far indicates, that this system can be utilized to track the origin of these specific compounds to specific plants or plant parts used to arrive at a therapeutic formulation. (As shown in FIG. 2)

### Example 3

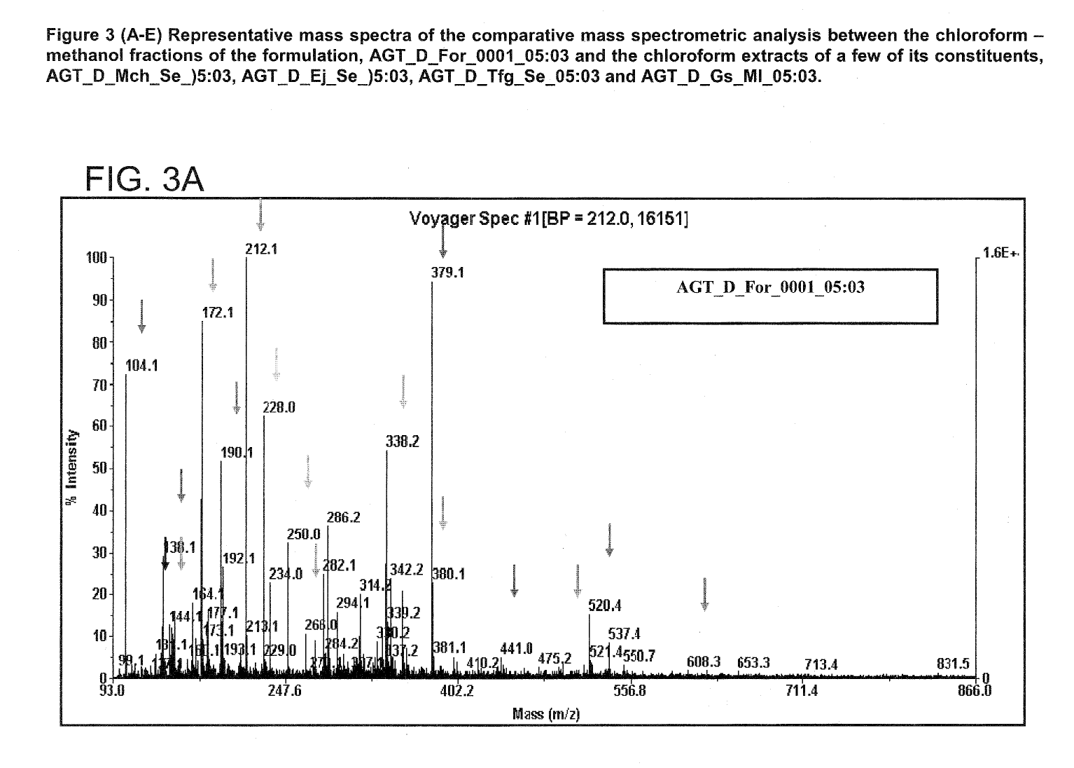

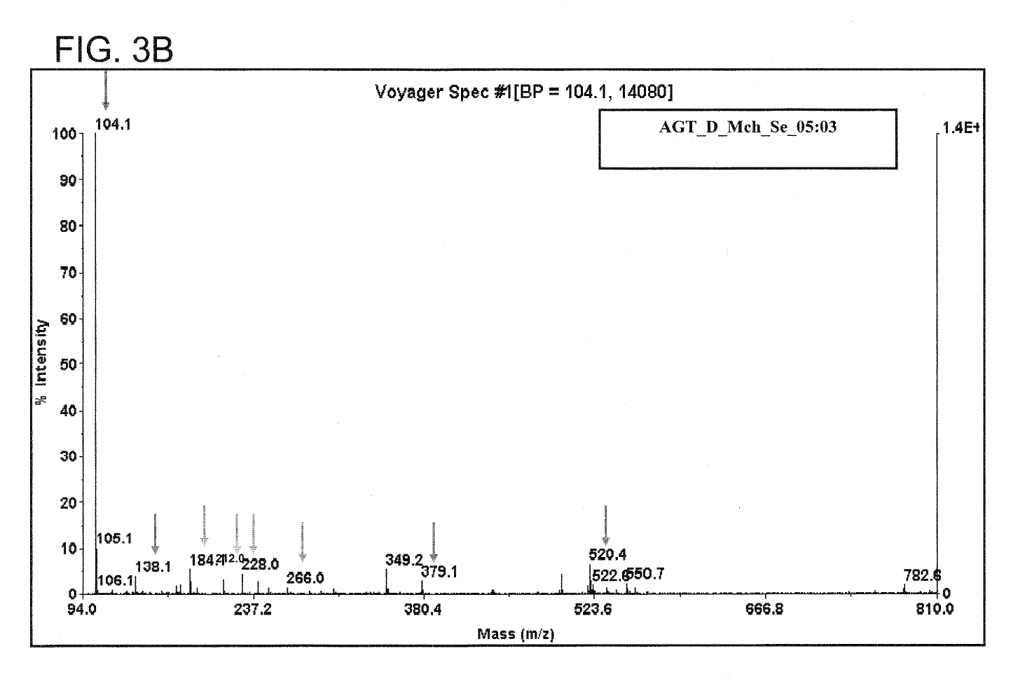

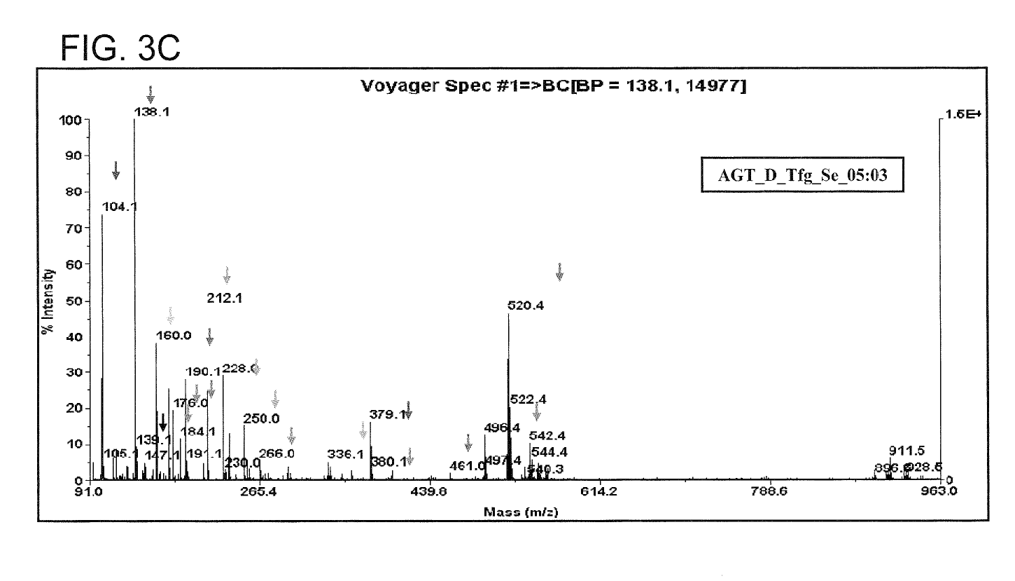

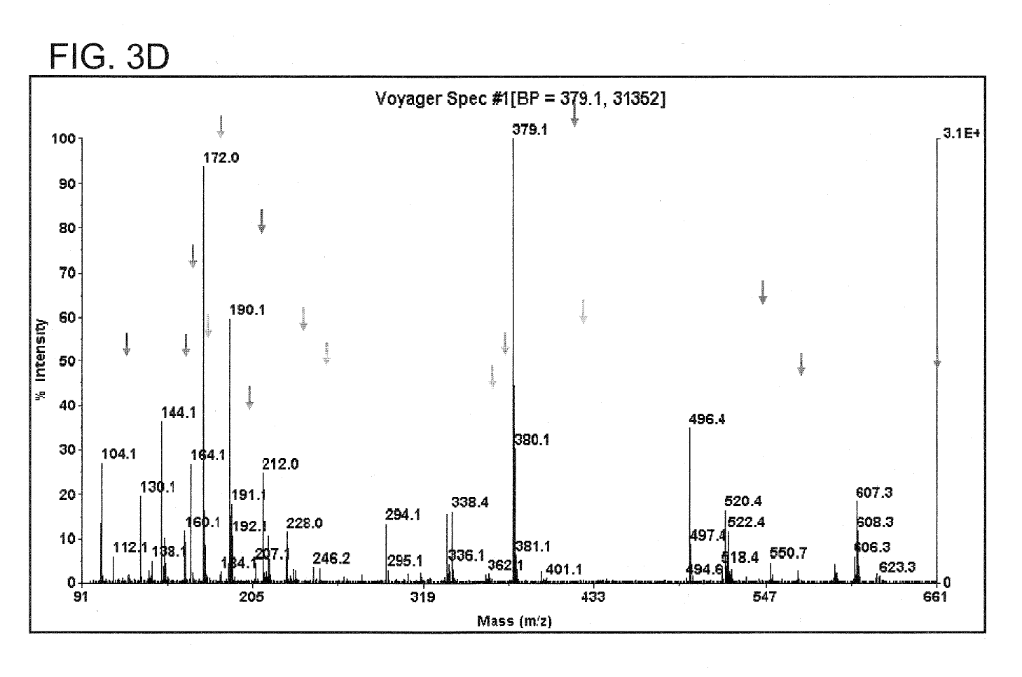

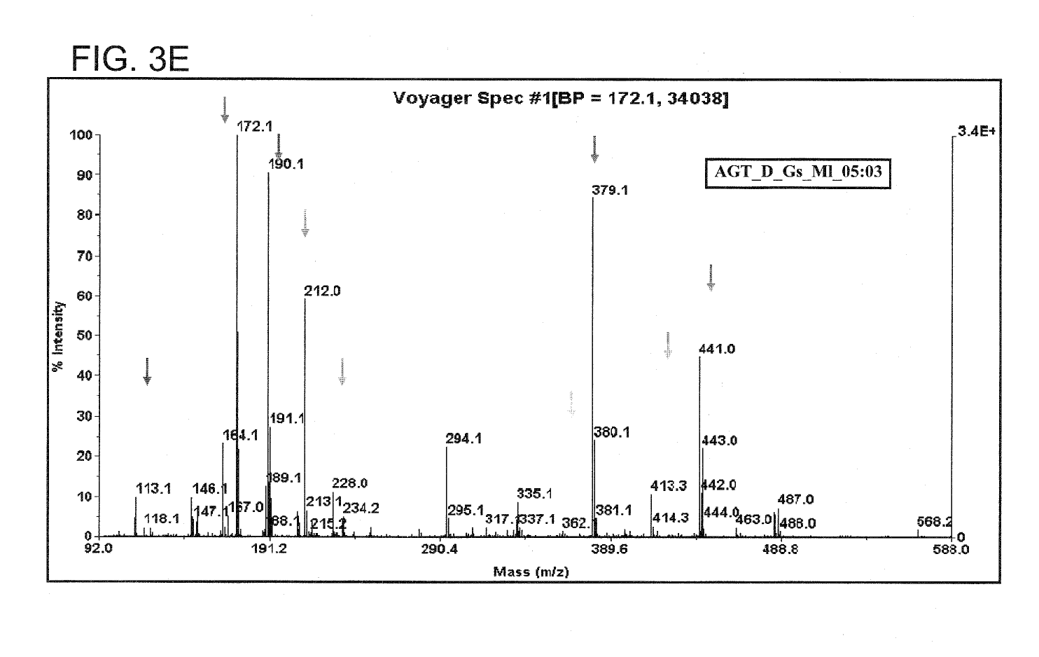

The basic alkaloids were extracted by method 2 and were profiled by HPLC and mass spectrometry. The comparative results of the mass spectrometry analysis the chloroform-methanol fractions of formulation AGT_D_For_{—}0001 05:03 and its constituents, AGT_D_Mch_Se_{—}05:03, AGT_D_Tfg_Se_{—}05:03, AGT_D_Ej_Se_{—}05:03, AGT_D_Gs_Ml_{—}05:03 reveals the common mass, m/z 104, 138, 172, 184, 336 and is suggestive of presence basic alkaloids and its precursors, which may have significant role in the treatment of diseases. As mentioned in example 2, the mass spectral peaks such as m/z-m/z 112, 155, 212, 286, 288, 338, 352.16, 496.38, 520.39, 623.48 that are present in formulation but are also uniquely present in few of the medicinal plant parts analyzed thus far indicates, that this system can be utilized to track the origin of these specific compounds to specific plants or plant parts used to arrive at a therapeutic formulation. (As shown in Table 4 & FIG. 3)

### Example 4

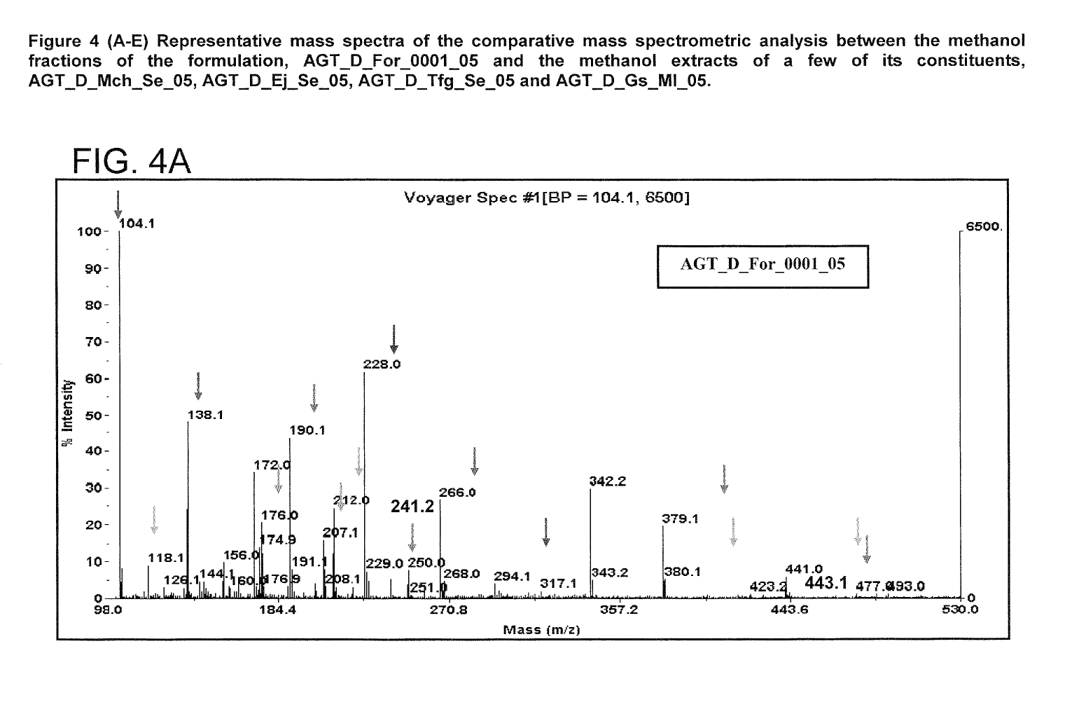

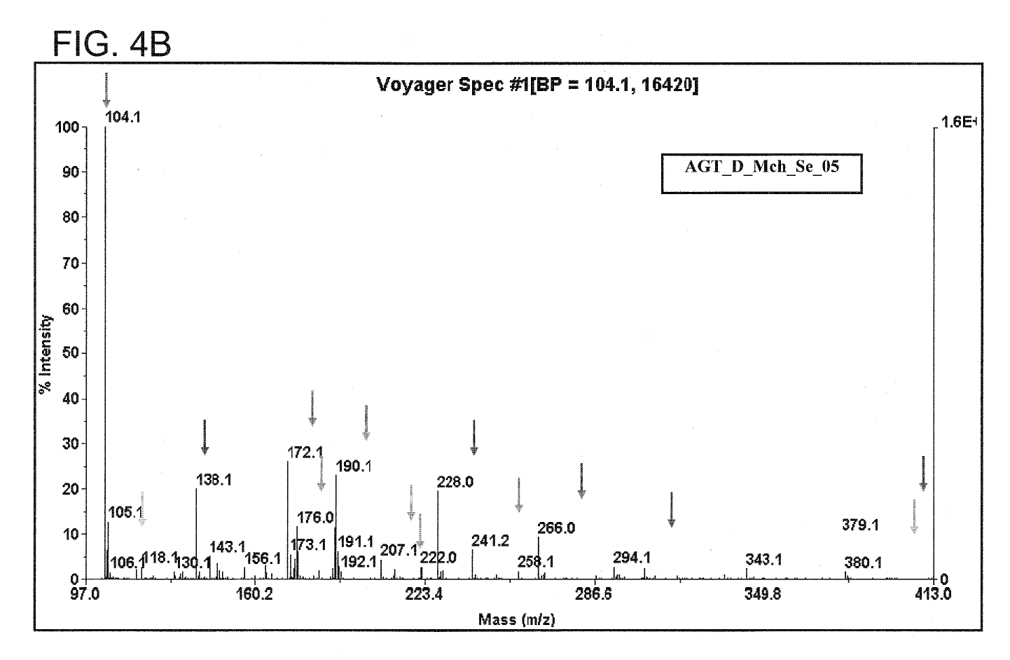

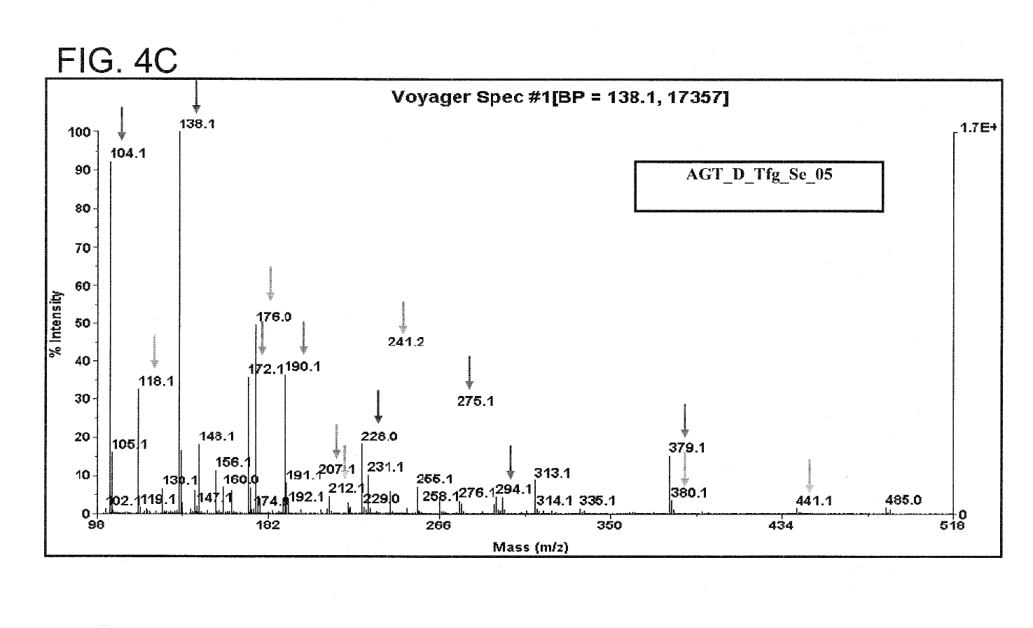

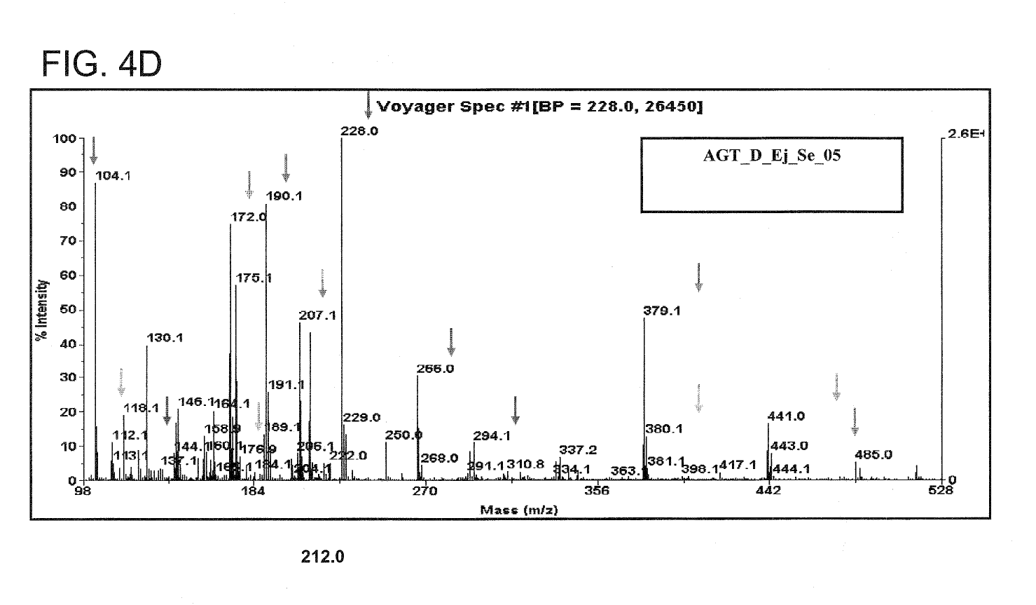

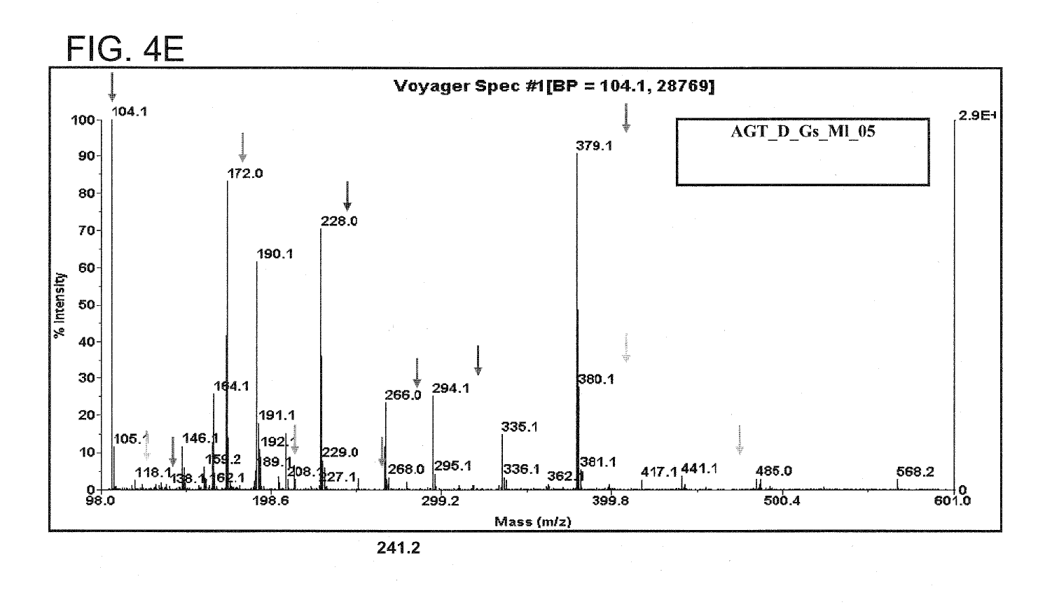

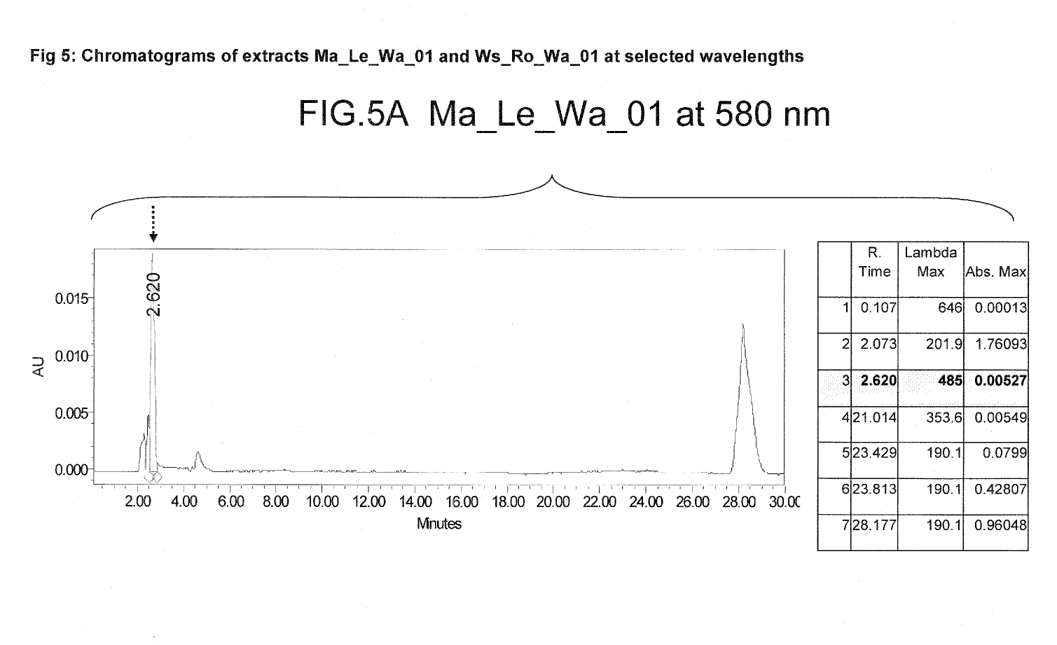

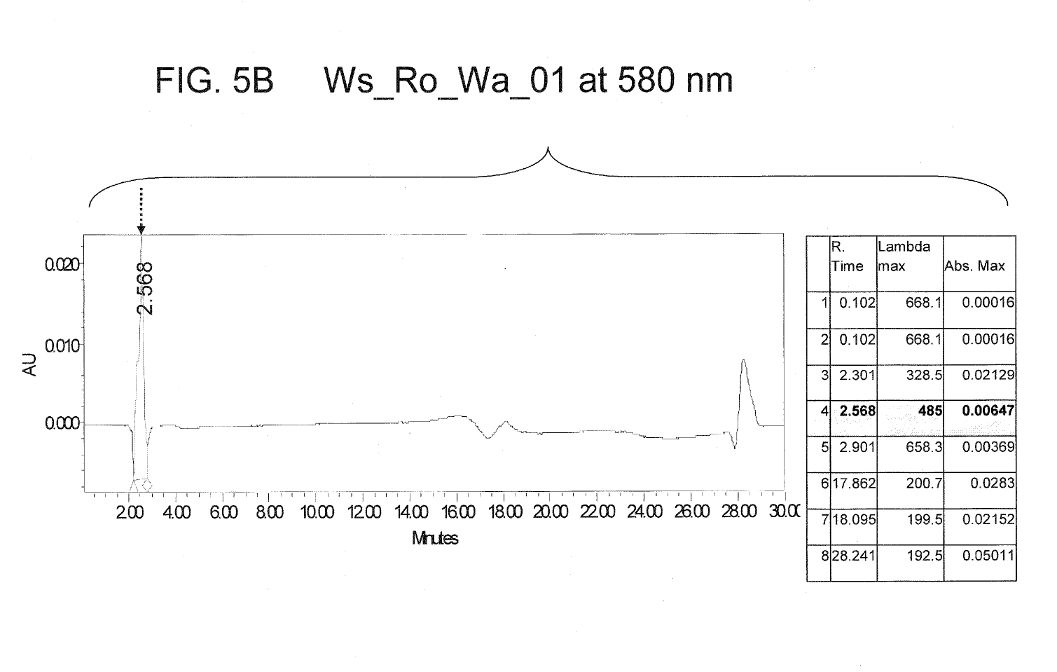

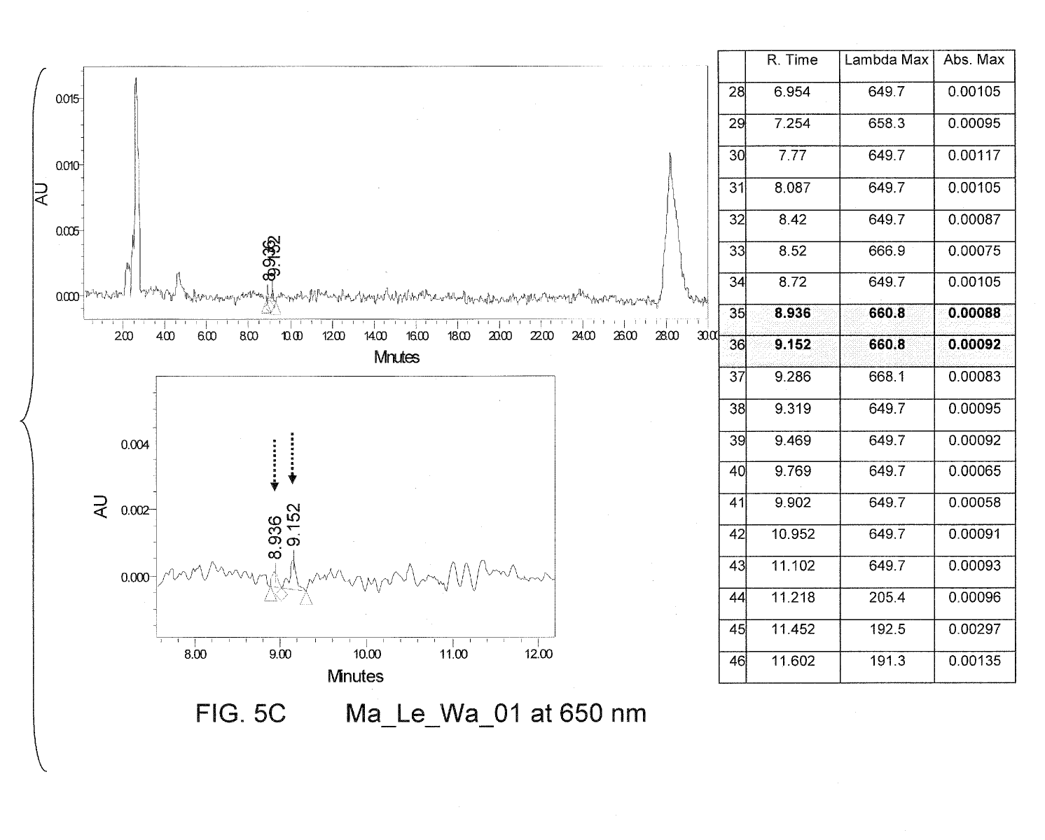

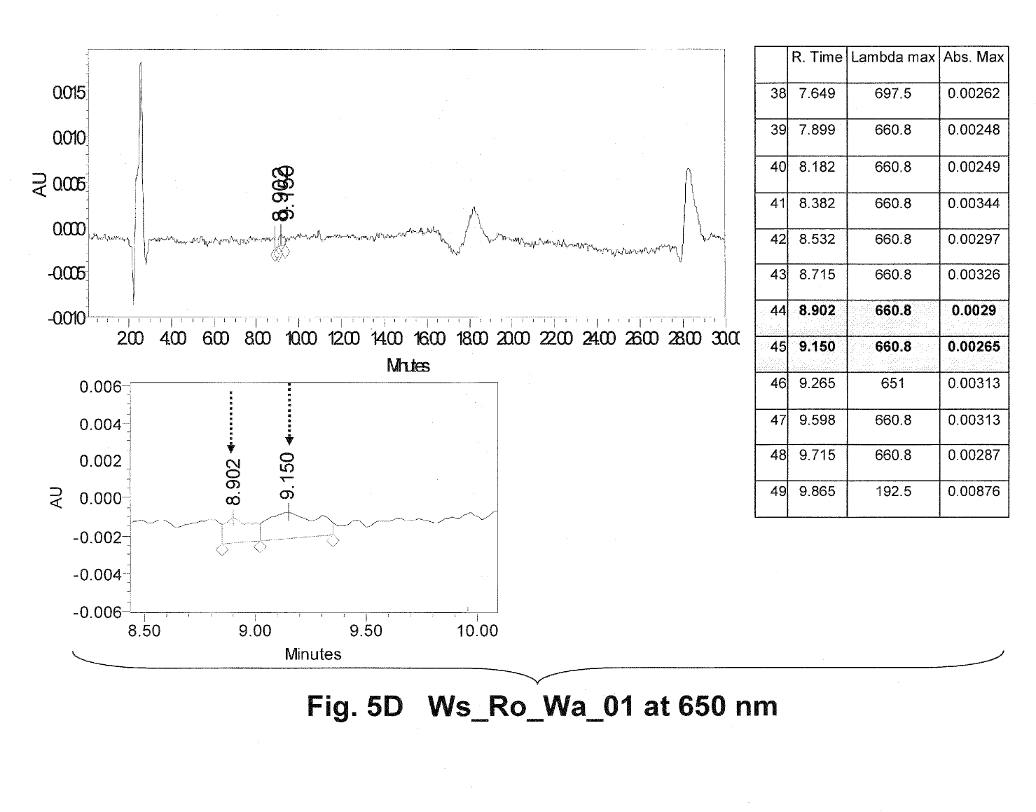

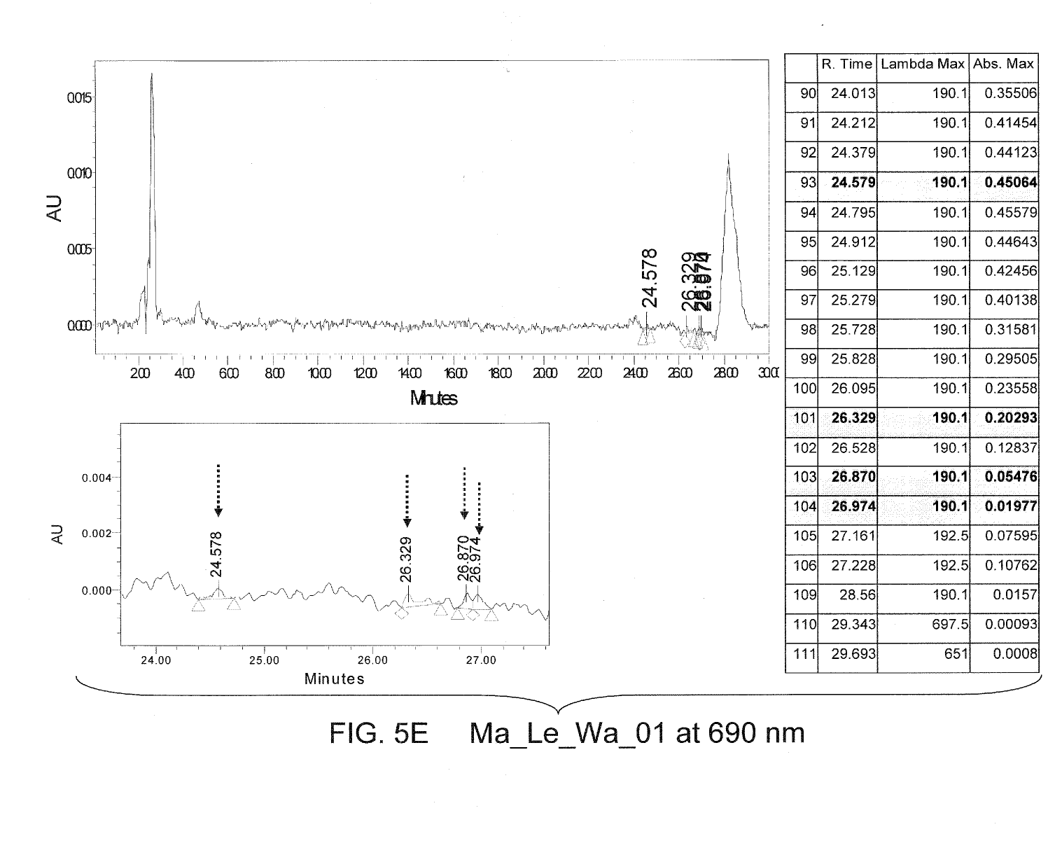

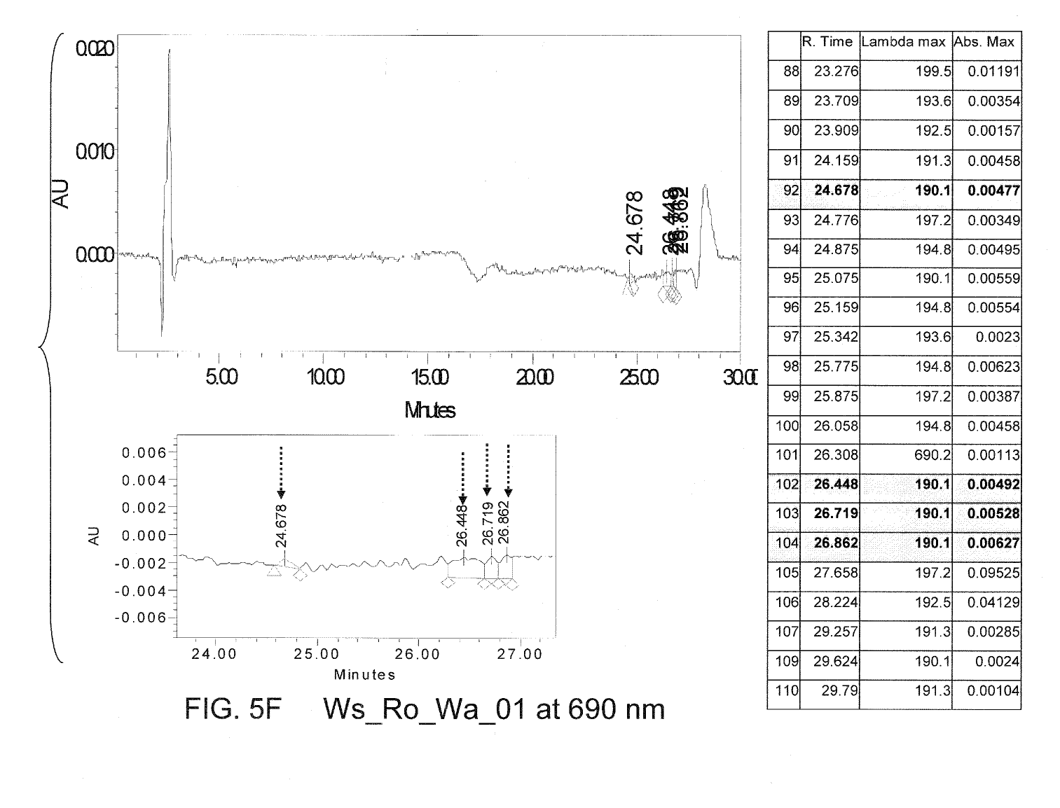

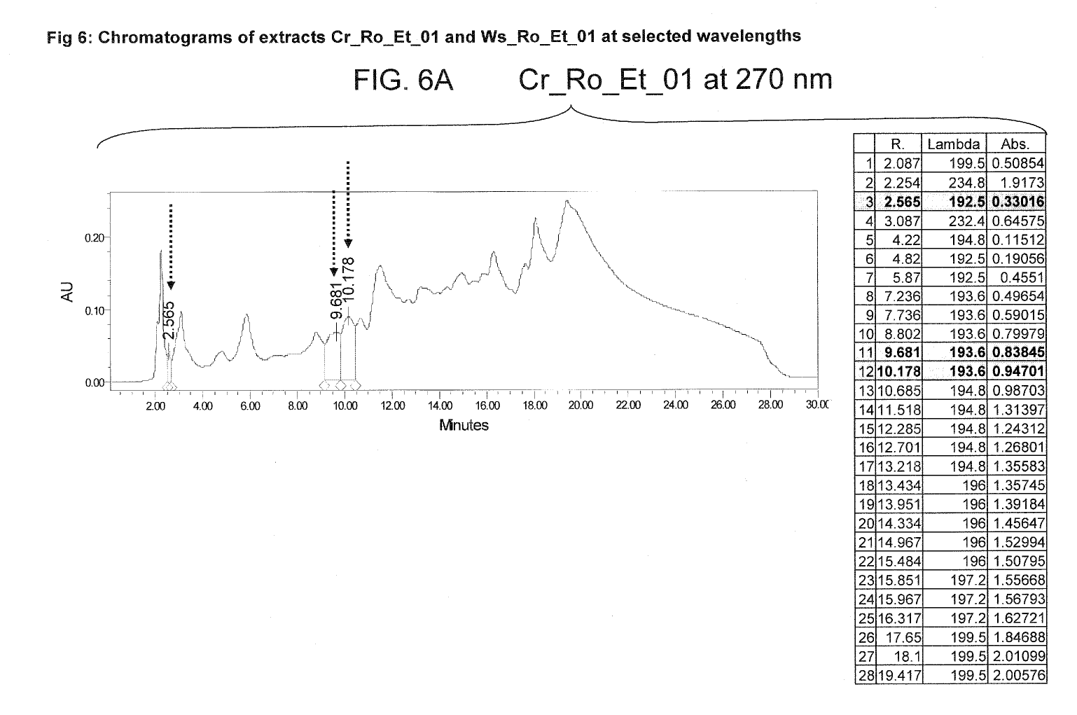

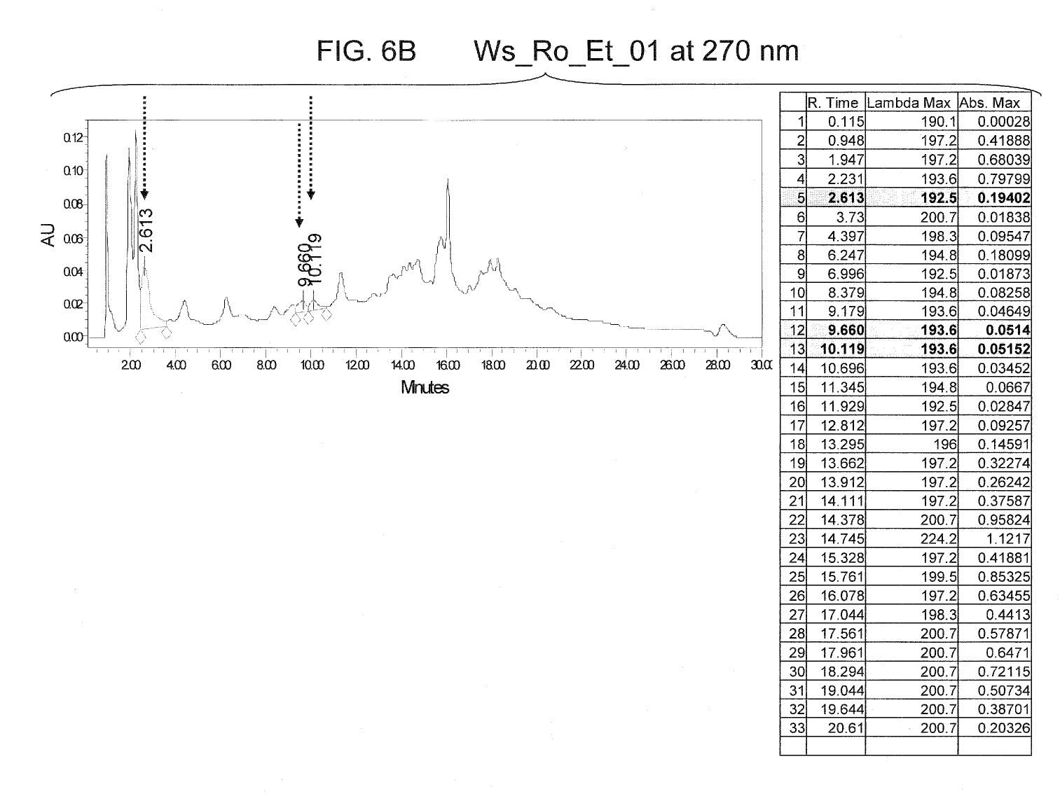

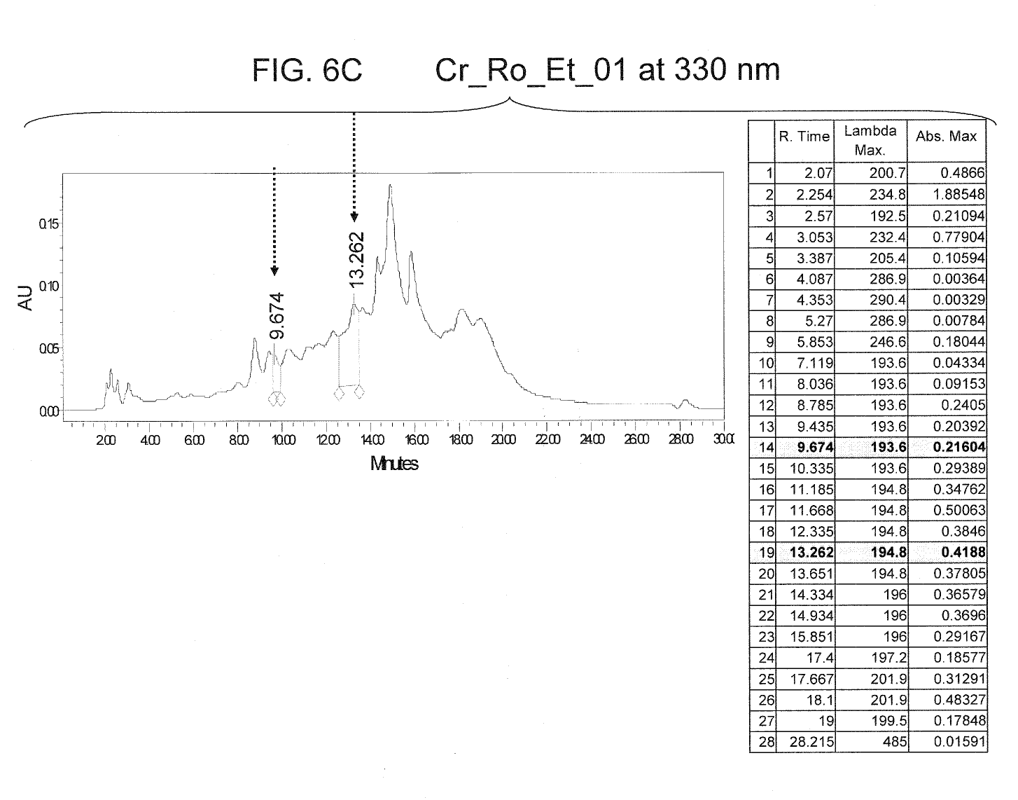

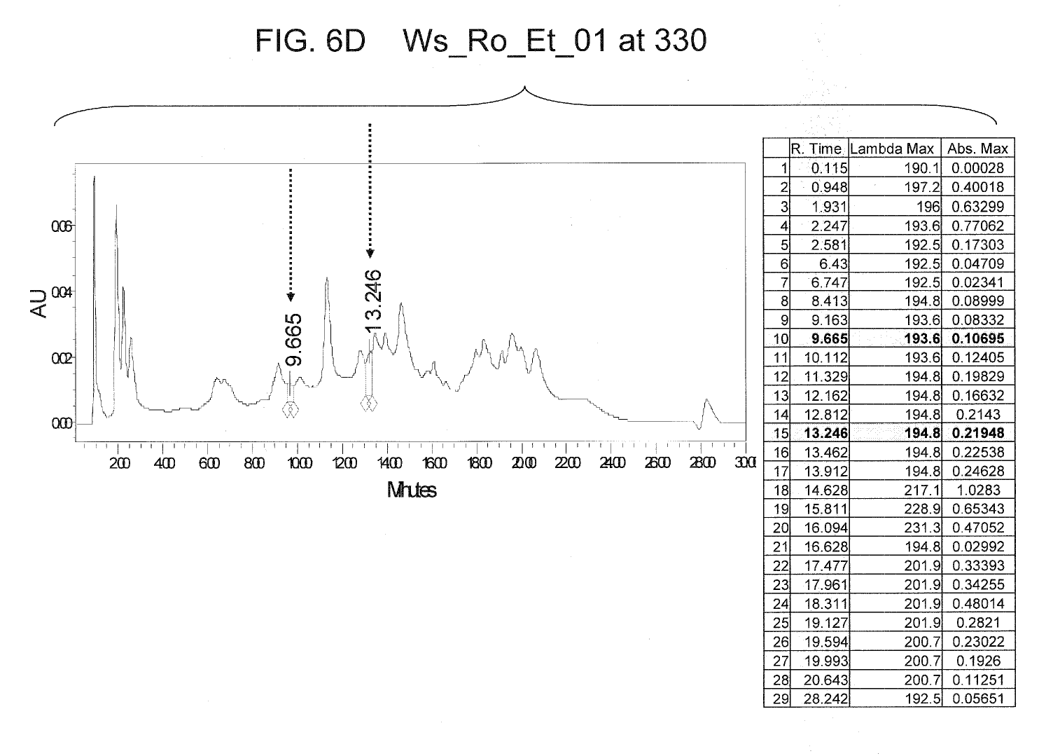

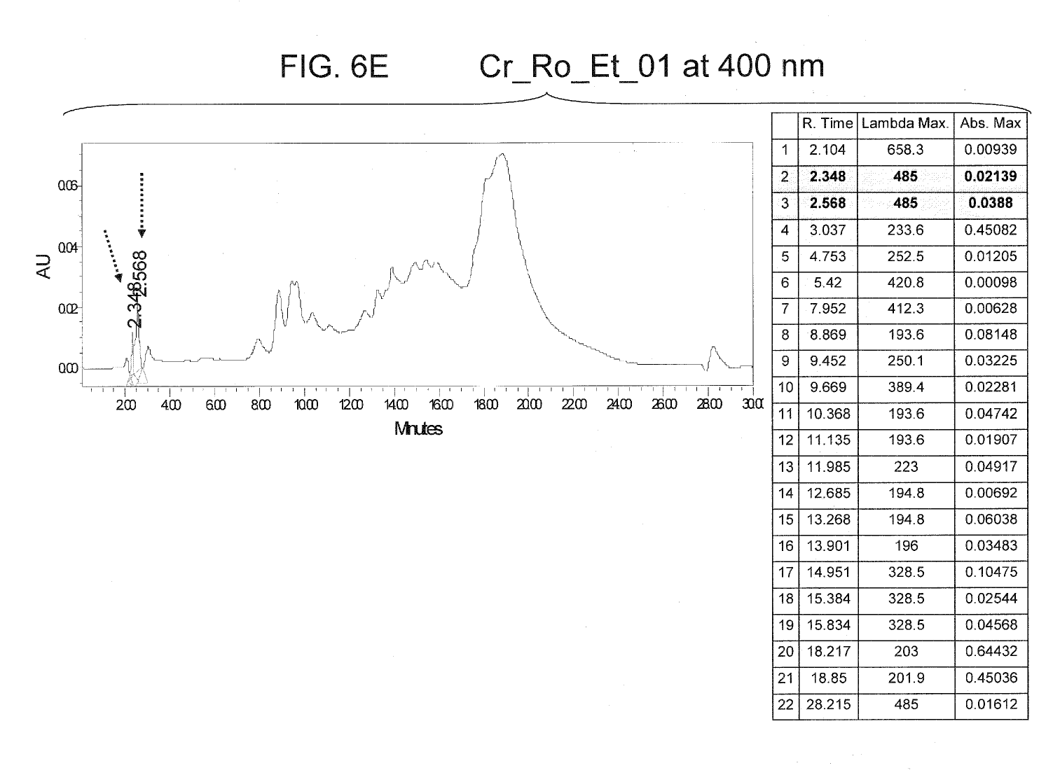

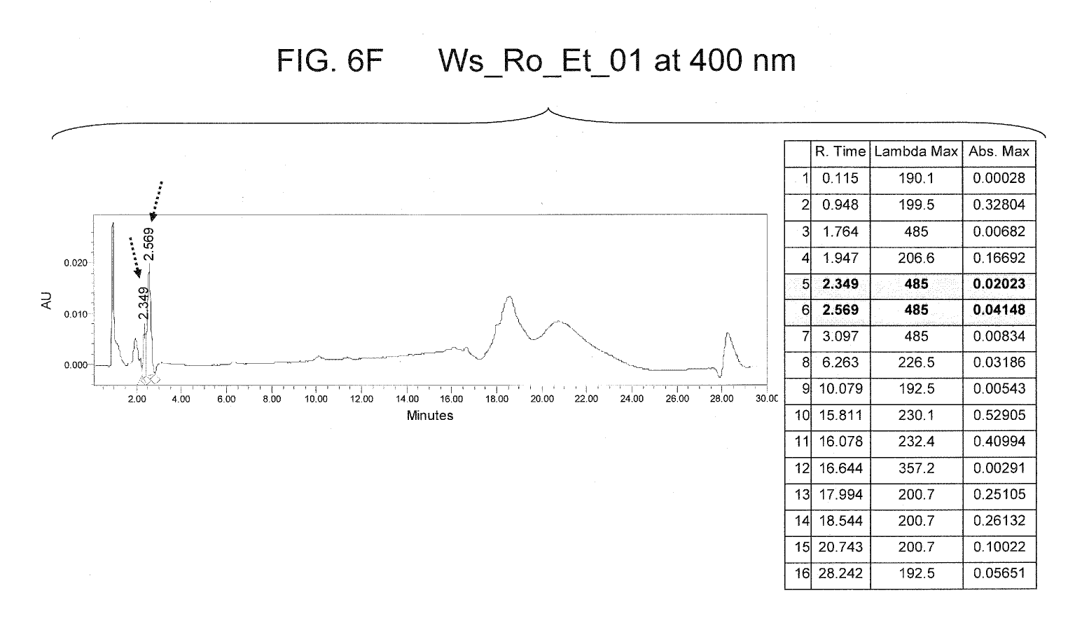

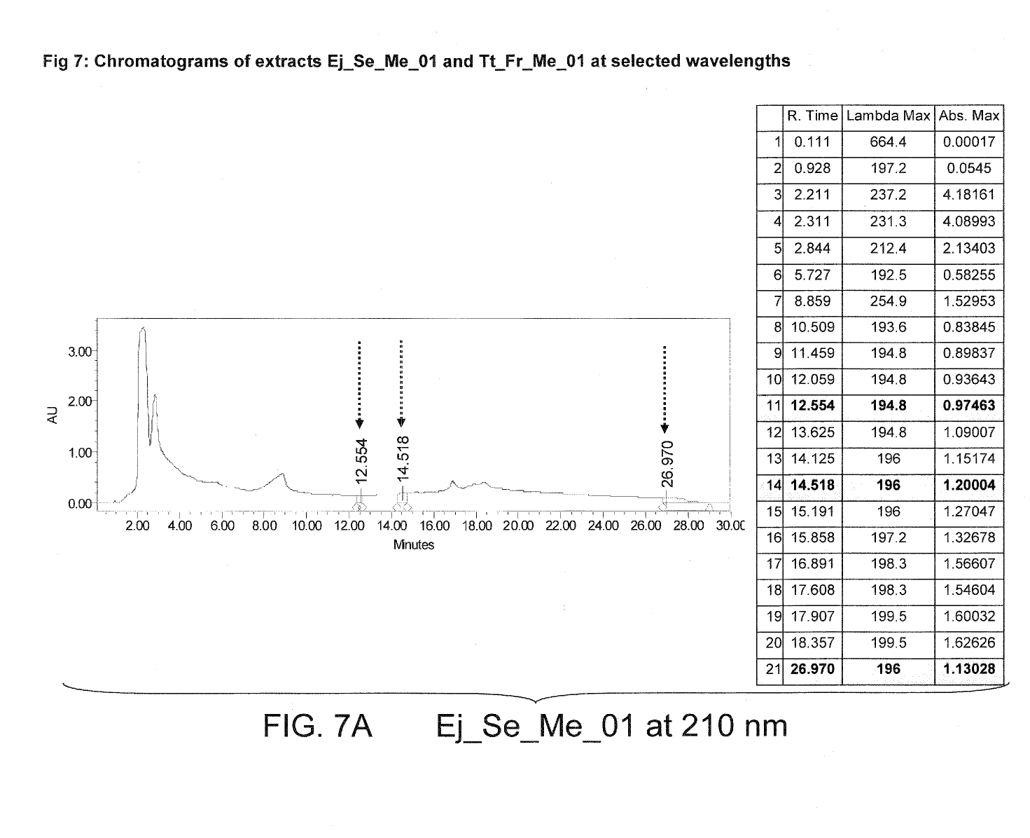

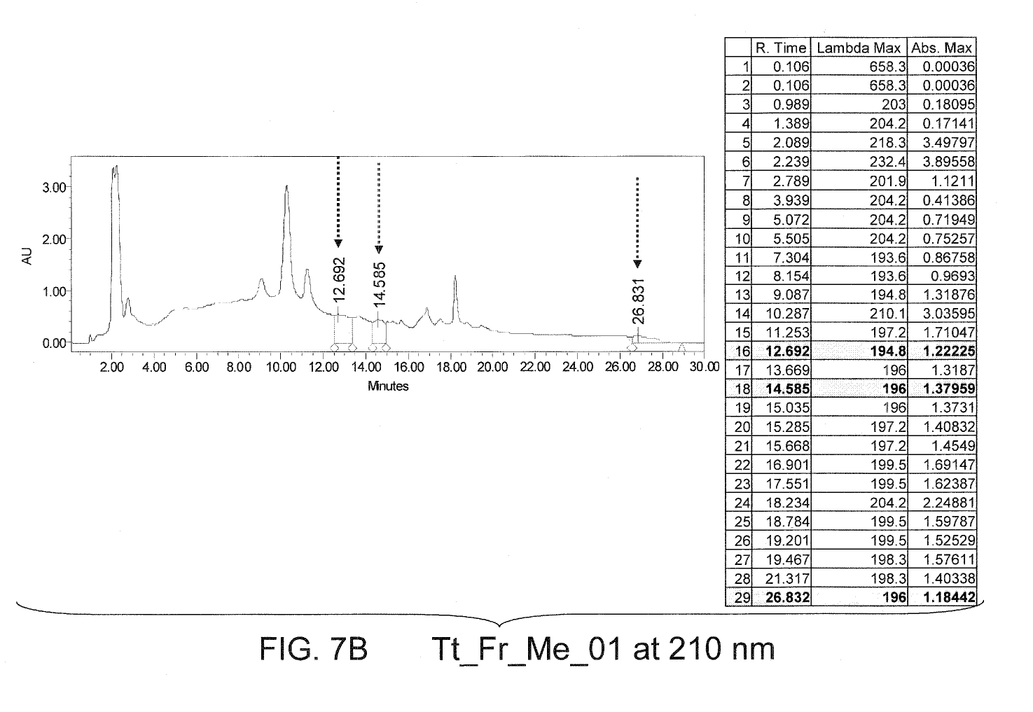

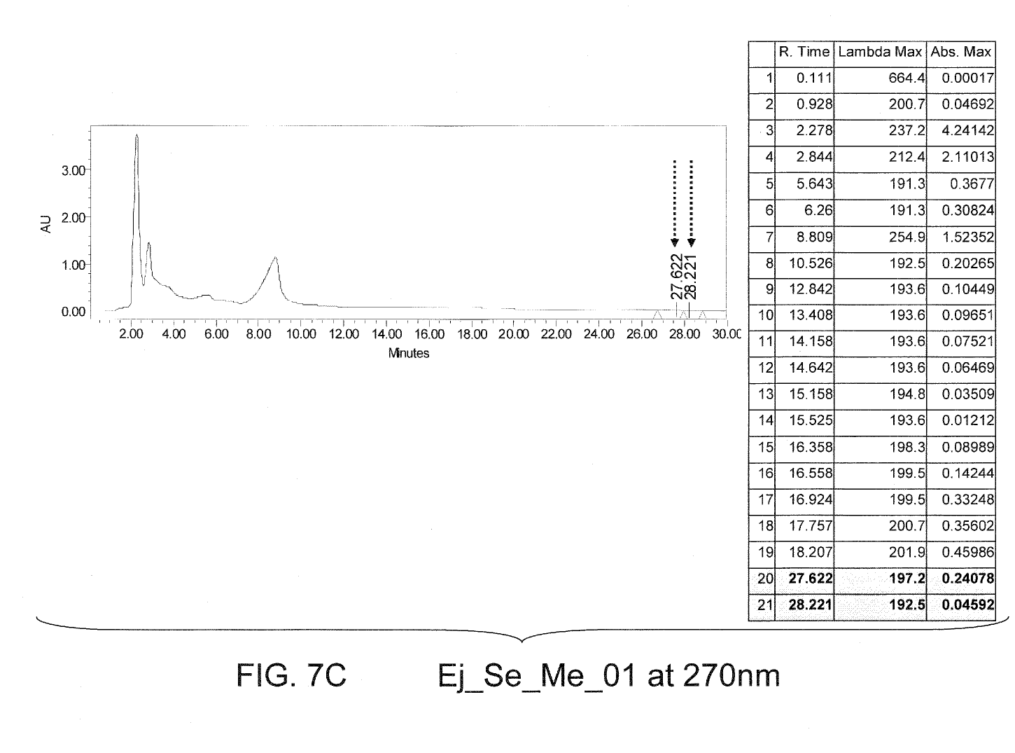

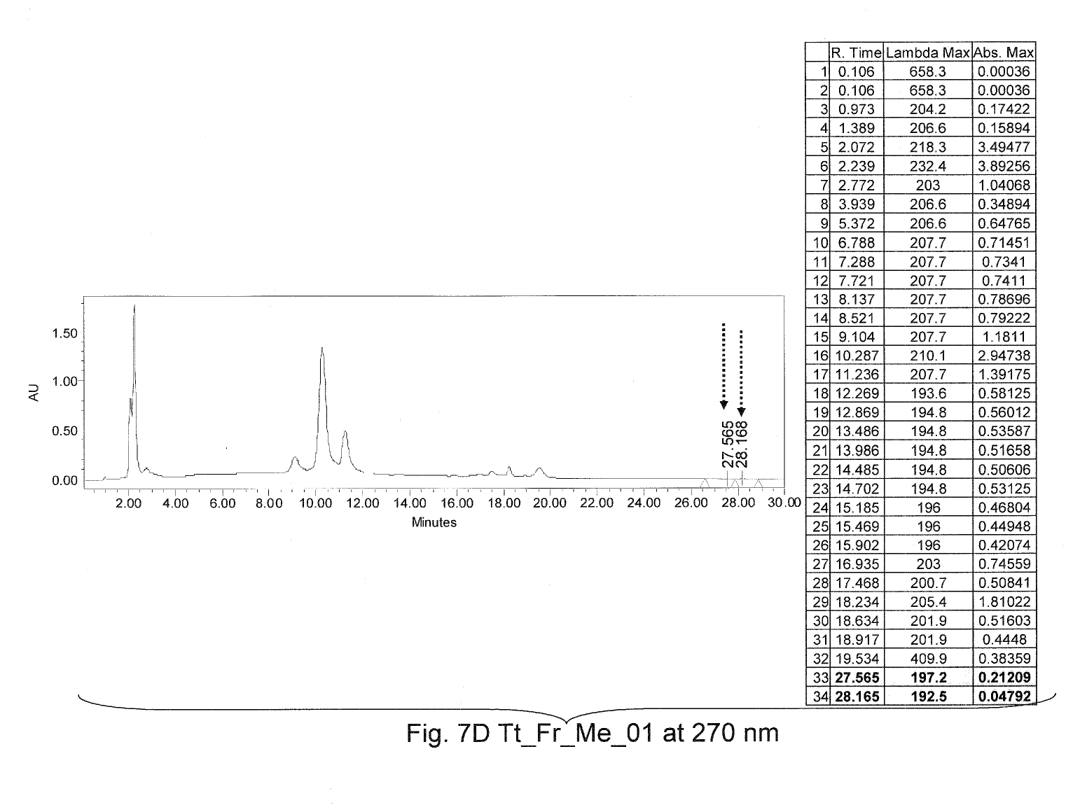

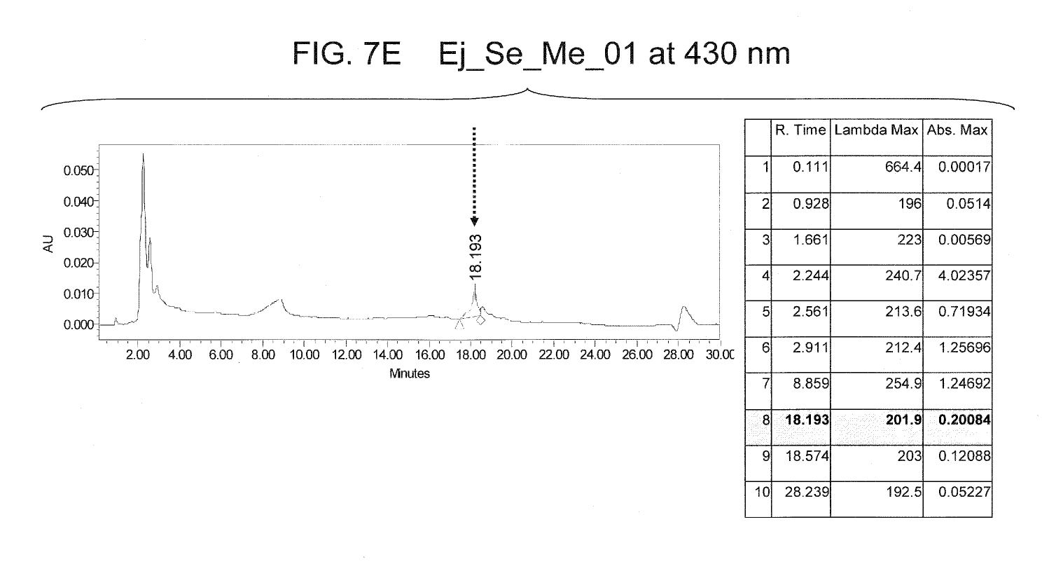

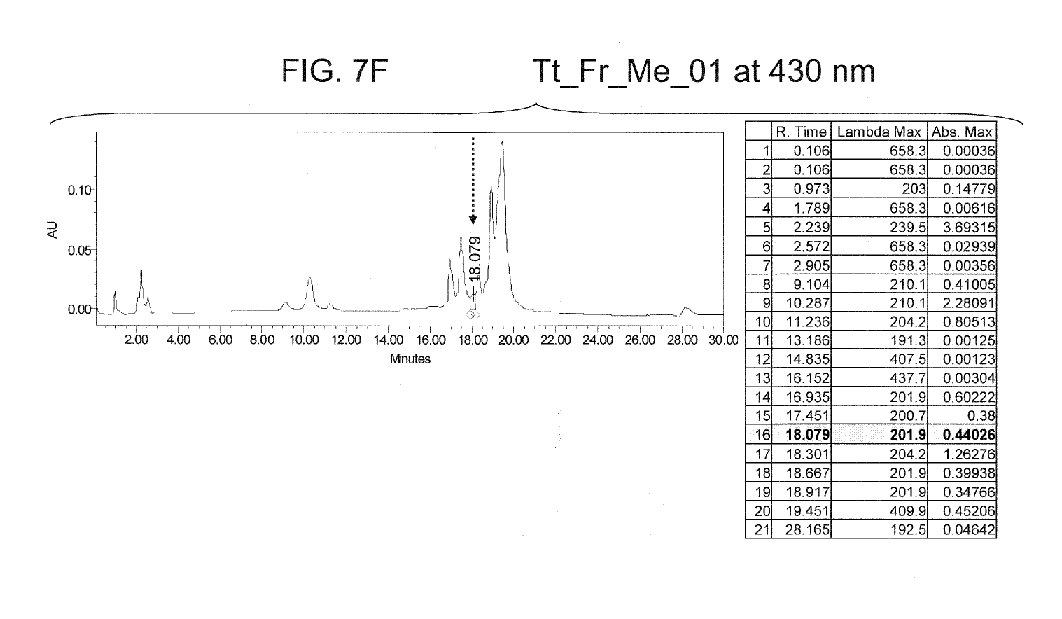

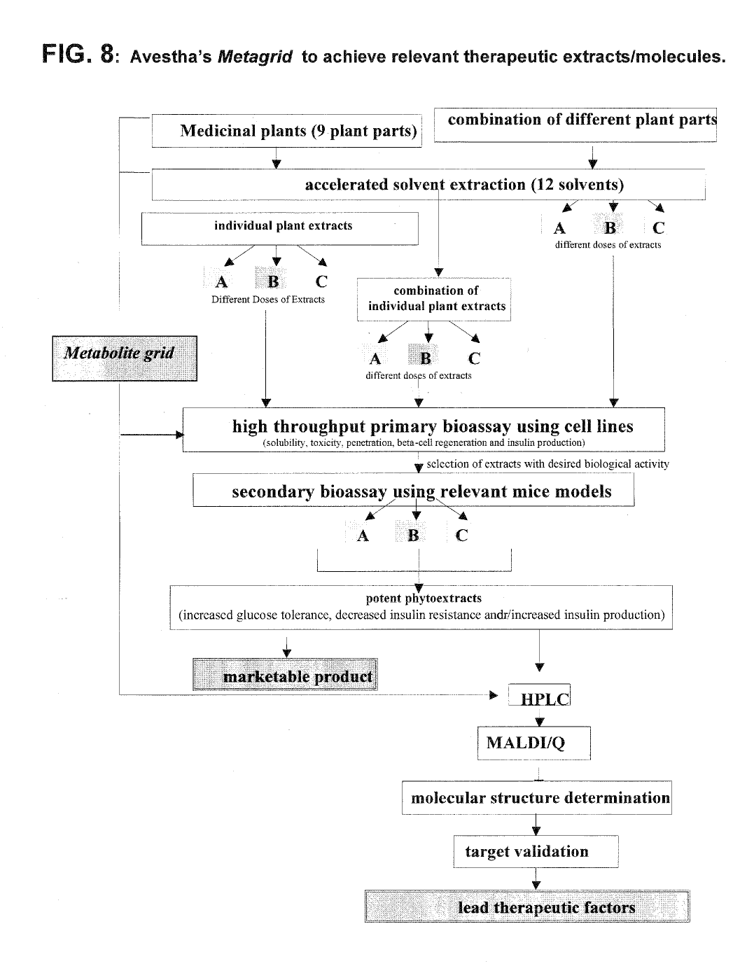

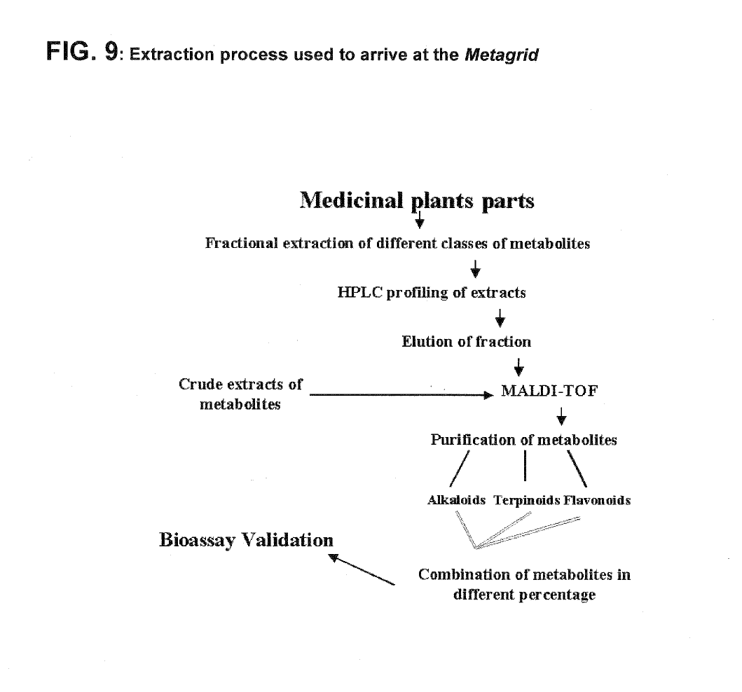

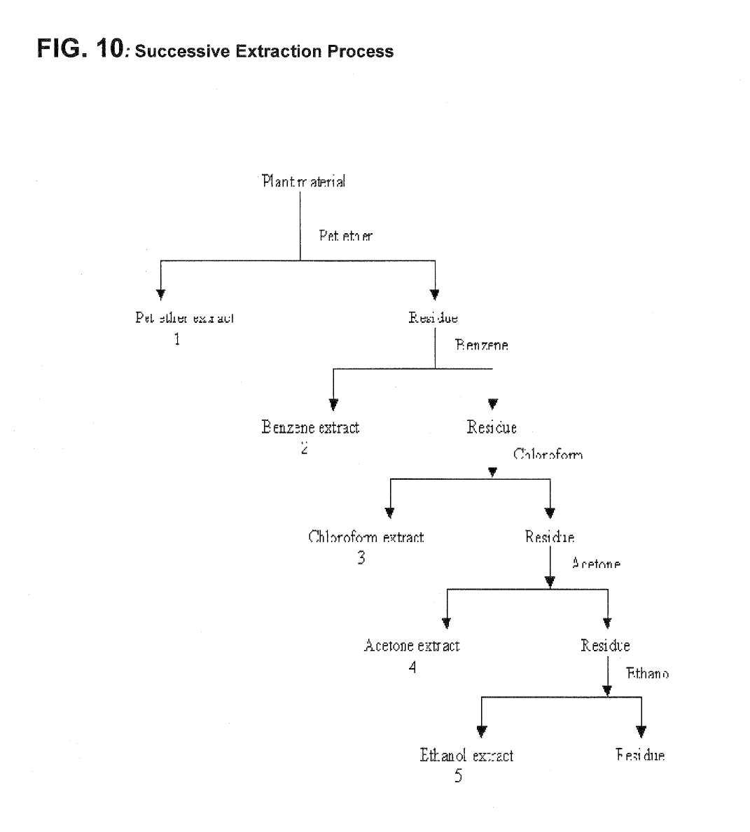

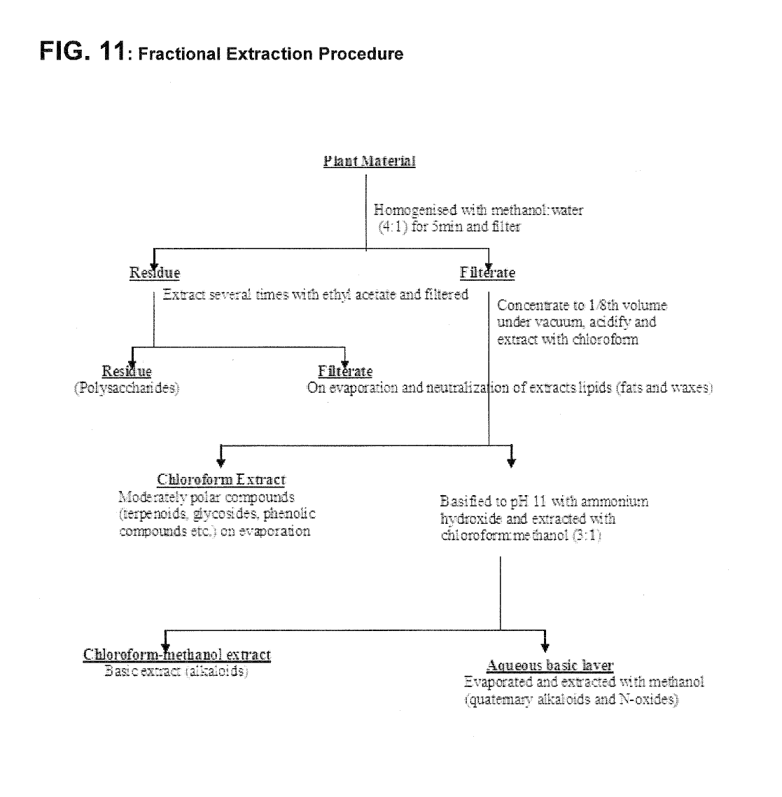

The quaternary alkaloids were extracted by method 2 and were profiled by HPLC and mass spectrometry. The comparative results of the mass spectrometry analysis of the methanolic fractions of formulation AGT_D_For_{—}0001_{—}05 and its constituent plants, AGT_D_Mch_Se_{—}05 AGT_D_Tfg_Se_{—}05, AGT_D_Ej_Se_{—}05 and AGT_D_Gs_Ml_{—}05 revealed the presence of common mass spectral peaks m/z 104, 172, 190, 212, 228, 294, 379 that are representative of quaternary alkaloids, N-oxides and, their precursors. As mentioned in example 2 and 3, the mass spectral peaks such as m/z-118.1, 138.06, 241.17, 250.1, 265.97, 296, 345, 441.05, 443.04, 492.98 that are present in formulation but are also uniquely present in few of the medicinal plant parts analyzed thus far indicates, that this system can be utilized to track the origin of these specific compounds to specific plants or plant parts used to arrive at a therapeutic formulation. (As shown in Table 5 & FIG. 4)

### HPLC Based Comprehensive Constituent Profiling and Metabolite Fingerprinting of Individual Plant Extracts:

The extracted fractions were subjected to HPLC using μ bondapak C₁₈ column (Waters Alliance 2695 Separation Module) to separate the constituent metabolites. The fractions were eluted using a combination (80:20, 60:40, 50:50, 40:60, 20:80) of methanol:water/acetonitrile:water. The gradient run was also carried out wherever required. 5-10 ul of sample was injected with flow rate of 1 ml/min and HPLC run was performed for 30 minutes. The detection was carried out on photodiode array and the analysis of the results was done with the help of Millennium™ software.

Comprehensive constituent profiling and metabolite fingerprinting of individual 51 individual plant extracts (see table 2) has been carried out. (See Table 6 and FIG. 5)

Comparative metabolite fingerprinting analysis has revealed the presence of both common and unique constituents that are present in individual plant extracts that have been extracted under similar conditions. (See Table 7)

The intuitive methodology used to arrive at the data (using a combinatorial matching of retention times and absorption maxima) summarized in table 7 is illustrated using a few representative instances. shown in FIGS. 6 & 7.

The extracts were randomly screened for metabolites by HPLC and mass spectrometry, the results reveal the group of common metabolites in most of the extracts and some unique molecular masses were also observed which would be further subjected to structural elucidation and characterization by MS″ and NMR. Furthermore, derivatisation of these characterized molecules will be carried out using the synthetic chemistry approach.

Carbon dioxide extraction procedure would be carried as against the conventional method of extraction, expecting more efficient extraction with less solvent consumption and shorter extraction time.

### Screening of Extracts for its Bioactivity:

51 (Fifty One) individual extracts and their combinations are ready for primary and secondary assay systems for Diabetes.

The extracts will be tested for primary assay:
a. using murine pancreatic islet cell lines (HIT, HIP, RIN, alpha-TC and beta-TC) to monitor the changes in levels of insulin-secretion in response to the treatment with defined phyto-extracts
b. changes in insulin-resistance will also be monitored in the murine adipocyte cell line-3T3-L1 in, response to the treatment with defined phytoextracts.

Secondary bioassay using mouse models will be conducted to validate the phytoextracts. Different kinds of mouse models will be used for this purpose
a. streptozotocin (STZ)/alloxan induce diabetic mouse.
b. Genetically modified mouse models (db/db, db/ob, and ob/ob)

The gene for ORCA-3, a jasmonate responsive APETALA2 (AP2) domain transcription factor may be isolated by transferred DNA activation tagging and expressed in these therapeutic plants expecting an enhanced expression of several metabolites biosynthetic genes and consequently, an increased accumulation of secondary metabolites of interest in the treatment of diabetes and allied disorders.

TABLE 1
List of medicinal plants used to arrive at the formulation,
AGT_D_For_0001

| Formulation | | |
|---|---|---|
| AGT_D_For_0001 | | Botanical name |
| 1 | Amlaki | Phyllanthus emblica |
| 2 | Guduchi | Tinospora cordifolia |
| 3 | Nimbha | Azadiractha indica |
| 4 | Jambu | Eugenia jambulana |
| 5 | Medhika | Trigonella foenum graceum |
| 7 | Haritaki | Terminalia chebula |
| 8 | Vibhitaki | Mucunapuriens |
| 9 | Haridra | Curcuma longa |
| 10 | Udumbara | Ficus glomerata |
| 11 | Bhumyamalaki | Phyllanthus niruri |
| 12 | Ashwagandha | Withania somenifera |
| 13 | Karavalli | Momordica charantia |
| 14 | Meshasringi | Gymnema sylvestre |
| 15 | Silajit | Euphorbia royleana |

TABLE 2
List of extracts from individual plants

| Extract ID | Plant name | Tissue | Solvent |
|---|---|---|---|
| Cr_Ro_Me_01 | Catharanthus roseus | Root | methanol |
| Cr_Ro_Et_01 | Catharanthus roseus | Root | ethanol |
| Cr_Ro_Ch_01 | Catharanthus roseus | Root | chloroform |
| Ej_Se_Me_01 | Eugenia jambolana | Seed | methanol |
| Ej_Se_Et_01 | Eugenia jambolana | Seed | ethanol |
| Ej_Se_Ch_01 | Eugenia jambolana | Seed | chloroform |
| Ej_Se_Pe_01 | Eugenia jambolana | Seed | petroleum ether |
| Ej_Se_Be_01 | Eugenia jambolana | Seed | benzene |
| Ej_Se_Et_01(20) | Eugenia jambolana | Seed | ethanol-20% |
| Ej_Se_Wa_01 | Eugenia jambolana | Seed | water |
| Eo_Fr_Me_01 | Emblica officinalis | Fruit | methanol |
| Eo_Fr_Ch_01 | Emblica officinalis | Fruit | chloroform |
| Gs_Le_Me_01 | Gymnema sylvestre | Leaf | methanol |
| Gs_Le_Et_01 | Gymnema sylvestre | Leaf | ethanol |
| Gs_Le_Ch_01 | Gymnema sylvestre | Leaf | chloroform |
| Gs_Le_Pe_01 | Gymnema sylvestre | Leaf | petroleum ether |
| Gs_Le_Be_01 | Gymnema sylvestre | Leaf | benzene |
| Gs_Le_Et_01(20) | Gymnema sylvestre | Leaf | ethanol-20% |
| Gs_Le_Wa_01 | Gymnema sylvestre | Leaf | water |
| Ma_Le_Me_01 | Melia azadirechta | Leaf | methanol |
| Ma_Le_Et_01 | Melia azadirechta | Leaf | ethanol |
| Ma_Le_Ch_01 | Melia azadirechta | Leaf | chloroform |
| Ma_Le_Pe_01 | Melia azadirechta | Leaf | petroleum ether |
| Ma_Le_Be_01 | Melia azadirechta | Leaf | benzene |
| Ma_Le_Hx_01 | Melia azadirechta | Leaf | hexane |
| Mc_Fr_Me_01 | Morinda citrifolia | Fruit | methanol |
| Mc_Fr_Et_01 | Morinda citrifolia | Fruit | ethanol |
| Mc_Fr_Ch_01 | Morinda citrifolia | Fruit | chloroform |
| Pm_Ba_Et-01 | Pterocarpus marsupium | Bark | ethanol |
| Pm_Ba_Wa-01 | Pterocarpus marsupium | Bark | water |
| Tc_Fr_Me_01 | Tinospora cardifolia | Fruit | methanol |
| Tc_Fr_Et_01 | Tinospora cardifolia | Fruit | ethanol |
| Tc_Fr_Ch_01 | Tinospora cardifolia | Fruit | chloroform |
| Tt_Fr_Me_01 | Tribulus teristris | Fruit | methanol |
| Tt_Fr_Et_01 | Tribulus teristris | Fruit | ethanol |
| Tt_Fr_Ch_01 | Tribulus teristris | Fruit | chloroform |
| Tt_Fr_Pe_01 | Tribulus teristris | Fruit | petroleum ether |
| Tt_Fr_Be_01 | Tribulus teristris | Fruit | benzene |
| Tfg_Se_Me_01 | Trigonella foenum | Seed | methanol |
| | graecum | | |
| Tfg_Se_Et_01 | Trigonella foenum | Seed | ethanol |
| | graecum | | |
| Tfg_Se_Ch_01 | Trigonella foenum | Seed | chloroform |
| | graecum | | |
| Tfg_Se_Pe_01 | Trigonella foenum | Seed | petroleum ether |
| | graecum | | |
| Tfg_Se_Be_01 | Trigonella foenum | Seed | benzene |
| | graecum | | |
| Tfg_Se_Et_01(20) | Trigonella foenum | Seed | ethanol-20% |
| | graecum | | |
| Tfg_Se_Wa_01 | Trigonella foenum | Seed | water |
| | graecum | | |
| Ws_Ro_Me_01 | Withania somnifera | Root | methanol |
| Ws_Ro_Et_01 | Withania somnifera | Root | ethanol |
| Ws_Ro_Ch_01 | Withania somnifera | Root | chloroform |
| Ws_Ro_Pe_01 | Withania somnifera | Root | petroleum ether |
| Ws_Ro_Be_01 | Withania somnifera | Root | benzene |
| Ws_Ro_Wa_01 | Withania somnifera | Root | Water |

Table 3 shows a representative mass spectral peak grid arrived at using a comparative mass spectral peak analysis approach that illustrates the components that are either common or unique to individual extracts.

| Molecular Masses | Molecular Masses | |
|---|---|---|
| of constituents | of components | |
| (m/z) common to | (m/z), unique to | Molecular Masses of |
| both methanolic | Methanolic | components (m/z), unique to |
| and ethanolic extracts | extracts | chloroform extracts |
| 104 | 203 | 190 |
| 112 | 267 | 336 |
| 176 | 337 | 349 |
| 184 | 534 | 390 |
| 212 | 594 | 520 |
| 228 | | 621 |
| 241 | | |
| 496 | | |
| 522 | | |
| 558 | | |
| 592 | | |
| 606 | | |

TABLE 4
Comparative results of the mass spectrometry analysis of the
chloroform-methanol fractions of formulation AGT_D_For_0001_05:03
and its constituents, AGT_D_Mch_Se_05:03,
AGT_D_Tfg_Se_05:03, AGT_D_Ej_Se_05:03 &
AGT_D_Gs_Ml_05:03 indicating the
common mass spectral peaks occurring both in
the formulation and the constituent plants masses (m/z)

| Common mass spectral | | |
|---|---|---|
| peaks occurring | | |
| both in the formulation and | Mass spectral peaks that are uniquely | |
| the constituent plants masses (m/z) | present in the constituent plants (m/z) | |
| 104 | 112 | Ej |
| 138 | 155 | Gs |
| 172 | 212 | Gs, Ej |
| 184 | 286 | Ej |
| 336 | 288 | Tfg |
| | 338 | Ej |
| | 352.16 | Mch, Ej |
| | 496.38 | Mch, Tfg |
| | 520.39 | Mch, Tfg |
| | 623.48 | Mch, Ej, Gs |

TABLE 5
The comparative results of the mass spectrometry analysis of the
methanolic fractions of formulation AGT_D_For_0001_05 and
its constituent plants, AGT_D_Mch_Se_05, AGT_D_Tfg_Se_05,
AGT_D_Ej_Se_05 and AGT_D_Gs_Ml_0.5

| Common mass spectral | | |
|---|---|---|
| peaks occurring | | |
| both in the formulation and the | Mass spectral peaks that are uniquely | |
| constituent plants masses (m/z)) | present in the constituent plants (m/z) | |
| 104.1 | 118.1 | Tfg |
| | 138.06 | Mch, Tfg |
| 172.05 | 241.17 | Mch, Tfg |
| 190.07 | 250.1 | Ej |
| 207.1 | 265.97 | Ej, Mch, Gs |
| 212.05 | 296 | Gs |
| 228.01 | 342 | Ej |
| 294.1 | 441.05 | Ej, Tfg, Gs |
| 379.1 | 443.04 | Ej, Gs |
| | 492.98 | Gs |

TABLE 6
Estimation of total number of constituents contained in a few of
the individual plant extracts that have been characterized using
HPLC-based metabolite fingerprinting.

| Extract | | Constituents in | | Total number |
|---|---|---|---|---|
| ID | | UV Range | Visible Range | of constituents |
| 1 | Eo_Fr_Me_01 | 123 | 603 | 726 |
| 2 | Cr_Ro_Me_01 | 232 | 674 | 906 |
| 3 | Ws_Ro_Me_01 | 247 | 590 | 837 |
| 4 | Tfg_Se_Me_01 | 176 | 604 | 780 |
| 5 | Ej_Se_Me_01 | 150 | 656 | 806 |
| 6 | Tt_Fr_Me_01 | 193 | 507 | 700 |
| 7 | Mc_Fr_Me_01 | 169 | 615 | 784 |
| 8 | Tc_Fr_Me_01 | 226 | 561 | 787 |
| 9 | Ws_Ro_Et_01 | 218 | 598 | 816 |
| 10 | Tfg_Se_Et_01 | 152 | 616 | 768 |
| 11 | Ws_Ro_Wa_01 | 159 | 618 | 777 |
| 12 | Ma_Le_Et_01 | 221 | 540 | 761 |
| 13 | Cr_Ro_Et_01 | 229 | 533 | 762 |
| 14 | Ej_Se_Et_01 | 109 | 600 | 709 |
| 15 | Gs_Le_Et_01 | 191 | 552 | 743 |
| 16 | Gs_Le_Me_01 | 237 | 557 | 794 |
| 17 | Ma_Le_Wa_01 | 114 | 643 | 757 |

Table 7 summarizes the comparative HPLC-based fingerprinting analysis done in the case of different plant extracts using a few representative instances (Ma_Le_Wa_{—}01 v/s Ws_Ro_Wa_{—}01; Ca_Ro_Et_{—}01 v/s Ws_Ro_Et_{—}01 and Ej_Se_Me_{—}01 v/s Tt_Fr_Me_{—}01).

| | | Total No. | Common | Unique |
|---|---|---|---|---|
| Extract ID | | of constituents | Constituents | constituents |
| 1 | Ma_Le_Wa_01 | 757 | 7 | 750 |
| | Ws_Ro_Wa_01 | 777 | | 770 |
| 2 | Ca_Ro_Et_01 | 762 | 41 | 721 |
| | Ws_Ro_Et_01 | 816 | | 775 |
| 3 | Ej_Se_Me_01 | 806 | 40 | 766 |
| | Tt_Fr_Me_01 | 700 | | 660 |

1-31. (canceled)

32. A screening method for arriving at a formulation, wherein the formulation comprises a combination of herbal extracts from medicinal plants of India, useful for treating diabetes, comprising the steps of:
a) providing one or more medicinal plants of interest;
b) mixing at least a part of the plant with methanol:water (about 4:1) to yield a water-methanol fraction and a first residue;
c) separating the water-methanol fraction from the first residue;
d) mixing the water-methanol fraction with chloroform to yield a chloroform extract, wherein the chloroform extract contains terpenoids, glycosides, phenolic compounds, or any combination thereof;
e) adjusting the pH of the chloroform extract to about 11;
f) mixing the chloroform extract with chloroform:methanol (about 3:1) to yield a chloroform-methanol extract and an aqueous basic layer;
g) separating the chloroform-methanol extract from the aqueous basic layer, wherein the chloroform-methanol extract contains alkaloids; mixing the aqueous basic layer with methanol to yield a methanol extract, wherein the methanol extract contains quaternary alkaloids and N-oxides;
h) subjecting the extract to a high performance liquid chromatography (HPLC), eluting the extract and creating a chemical profile for the extract bioactivity following HPLC, wherein the extract is selected from the chloroform extract, the chloroform-methanol extract, the methanol extract, or a combination thereof;
i) contacting the extract with pancreatic cells that secrete insulin;
j) determining a level of insulin secretion of the pancreatic cells; wherein the determination is optionally made at multiple times to monitor the change over time; wherein a decrease of the level of insulin secretion indicates that the extract is useful for treating diabetes; and
k) selecting extracts useful for treating diabetes and combining the selected extracts to arrive at a formulation comprising a combination of herbal extracts useful for treating diabetes.

33. (canceled)

34. The method, according to claim 32, further comprising the following steps after step (d):
l) mixing the first residue with ethyl acetate to yield an ethyl acetate extract and a second residue; and
m) separating the ethyl acetate extract from the second residue, wherein the second residue contains polysaccharides and the ethyl acetate extract contains lipids.

35-37. (canceled)

38. The method according to claim 32, wherein the plant is selected from the group consisting of Phyllanthus emblica, Azadiractha indica, Terminalia chebula, Mucunapuriens, Curcuma longa, Ficus glomerata, Phyllanthus niruri, Momordica charantia, Euphorbia royleana, Catharanthus roseus, Eugenia jambolana, Emblica officinalis, Gymnema sylvestre, Melia azadirechta, Morinda citrifolia, Pterocarpus marsupium, Tinospora cardifolia, Tribulus teristris, Trigonella foenum graecum, and Withania somnifera.

39. The method according to claim 32, wherein the part of the plant is selected from the group consisting of roots, seeds, fruits, barks, leaves, and any combination thereof.
